(19) United States
(12) Patent Application Publication
(10) Pub. No.: US 2020/0197462 A1
(43) Pub. Date: Jun. 25, 2020

(54) NOVEL CANNABIS LINES AND EXTRACTS FOR TREATING SKIN DISORDERS

(71) Applicants: Pathway RX Inc., Lethbridge (CA); Inplanta Biotechnology Inc., Lethbridge (CA)

(72) Inventors: Olga Kovalchuk, Lethbridge (CA); Dongping Li, Lethbridge (CA); Rocio del Carmen Rodriguez-Juarez, Lethbridge (CA); Anna Kovalchuk, Lethbridge (CA); Dwight Darryl Hudson, Raymond (CA); Igor Kovalchuk, Lethbridge (CA)

(21) Appl. No.: 16/713,029

(22) Filed: Dec. 13, 2019

Related U.S. Application Data
(60) Provisional application No. 62/781,647, filed on Dec. 19, 2018.

(51) Int. Cl. A61K 36/185; A01H 5/12; A01H 6/28

(52) U.S. Cl. CPC A61K 36/185; A01H 6/28; A01H 5/12

(57) ABSTRACT

The present invention provides cannabis lines, extracts and methods for the treatment of inflammatory skin disorders such as psoriasis, eczema, melanoma skin cancer, and others. The method includes generation of unique lines, whole plant extract preparation, treating human 3D skin tissues and disease models with extracts in amount sufficient to modulate gene expression in the skin tissues. The modulation of gene expression then results in a reduction of the disease state-associated changes or aspects thereof in the treated skin tissues.

### FIELD OF THE INVENTION

The present invention relates generally to products and methods for treating skin, and more specifically to methods and products for treating skin from cannabis and hemp plants.

### BACKGROUND OF THE INVENTION

Skin disease is the fourth leading cause of disability worldwide [1]. Affecting millions of people, chronic inflammatory skin disorders—eczema, dermatitis, psoriasis as well as non-melanoma skin cancer (NMSC) are serious chronic conditions that result in significant morbidity and economic burden [2]. Two common inflammatory skin diseases, eczema and psoriasis, result from a complex interplay of genetic, immunologic, microbial and environmental factors.

Atopic dermatitis (AD) is the most common type of eczema, affecting between 20-30% of children and 2-10% of adults. It is characterized by itching and rush that commonly affect the face, back of the knees, wrists, hands, feet or other areas of the body. AD-affected areas are scaly, dry, thickened, and exhibit altered pigmentation.

AD is often characterized by onset during infancy or early childhood, intense pruritus and a chronic, relapsing course. Atopic dermatitis in infants typically involves commonly involves the cheeks, scalp, and extensor surfaces of the extremities in infants, shifting to chronic, inflammatory, lichenified plaques. The pathogenesis of AD is multifactorial and involves: (1) epidermal barrier dysfunction; (2) immune dysregulation; and (3) microbial dysbiosis [3]. A defective epidermal permeability barrier has been widely recognized as an important element in the pathogenesis of AD.

Eczema can flare up and subside, and symptoms can range from mild to very severe, negatively impacting quality of life. Some complications of eczema include skin infections, neurodermatitis, and eye complications.

While not curable, AD is managed by trigger avoidance, regular emollient use and anti-inflammatory therapies. Acute flares are typically treated with topical corticosteroids or topical calcineurin inhibitors to achieve disease control. In moderate to severe or refractory disease, treatment options include phototherapy, systemic steroids, immunomodulatory therapies (i.e., methotrexate, cyclosporine) or biologics.

Psoriasis is a chronic inflammatory skin disease with a worldwide prevalence of ˜2% [4]. Between 1 and 3% of the USA population and up to 900,000 Canadians will develop psoriasis in their lifetime. Psoriasis is caused by an overactive immune system, whereby the key manifestations include inflammation, flaking, and appearance of white, silvery, or red patches of skin [5-7].While the clinical manifestations of psoriasis are varied, plaque-type psoriasis (or psoriasis vulgaris) is the most common form. Plaque-type psoriasis is characterized by well demarcated, erythematous plaques with silvery-white scales, commonly located on the scalp, trunk and extensor surfaces of the extremities (i.e., elbows and knees). It has a bimodal age of onset (16-22 vs. 57-60 years of age) and affects both sexes equally. Psoriasis represents a multi-system inflammatory disorder rather than a purely dermatologic disease [4]. Some individuals with psoriasis can also develop psoriatic arthritis. Disease is characterized by remissions and exacerbations. The impact of psoriasis on psychological and mental health should not be underestimated as patients also have an increased prevalence of depression, anxiety and suicidal ideation.

Psoriasis results from complex interactions between the immune system, psoriasis-associated susceptibility loci and multiple environmental factors. In the past 20 years, research has confirmed that psoriasis is primarily mediated by pathogenic T cells that produce high levels of IL-17 in response to IL-23. Understanding the central role for the IL-23/IL-17 signaling pathway in the development of psoriasis has led to a major paradigm shift in the pathogenic model for this condition. The activation and upregulation of IL-17 in pre-psoriatic skin produces a self-amplifying, feed-forward inflammatory response in keratinocytes that leads to the development of mature psoriatic plaques by inducing uncontrolled keratinocyte proliferation, abnormal differentiation and leukocyte recruitment into the skin [8]. Psoriasis and AD exhibit markedly aberrant gene expression. Psoriasis as well as AD are characterized by keratinocyte hyperproliferation of epidermis, and hyperproliferation-associated keratins K6, K16, and K17 are considered to be the hallmarks of psoriasis [9]. Serpins (especially SERPINB3/B4) are expressed in skin fibroblasts and keratinocytes, and their overexpression is induced by inflammation and hypoxia, further promoting an inflammatory loop was shown to underlie the chronic nature of psoriasis. Serpins have been proposed as potential psoriasis biomarkers [10]. Pro-inflammatory S100 family important in psoriasis [11]. S100A7 gene (psoriasin) is overexpressed in psoriasis and may be involved in its pathogenesis [12]. S100A8 and S100A9 were also shown to be significantly upregulated is psoriasis [13]. In-depth analysis of the psoriatic transcriptome led to identification of numerous regulated genes that include members of the Small Proline Rich Region (SPRR), Late Cornified Envelope (LCE), Involucrin (IVL), NICE-1, Cornulin (CRNN), S100, members of interferon pathway, various cytokines and chemokines, and others [15]. These genes and proteins constitute excellent therapeutic targets.

Psoriasis is characterized by remissions and exacerbations, and its management depends on disease severity. For mild to moderate psoriasis, first-line treatment commonly includes topical steroids, vitamin D3 analogues and/or combination products. Topical retinoids, anthralin, tar and other preparations are less commonly used. Moderate to severe disease typically requires more aggressive treatment with phototherapy, systemic immunomodulators (i.e., methotrexate, cyclosporine or systemic retinoids), small molecules (i.e., apremilast) or biologics such as anti-TNFα and anti-IL-12/23 antibodies. All of those treatments have very significant side effects, and often rather limited effectiveness [14].

Many psoriasis and AD treatments have a variety of potential side effects including bone marrow suppression, risk of infection, hepato- and nephrotoxicity and non-melanoma skin cancer. There is an urgent need to develop minimally invasive, easy-to-use, skin-specific therapies having little to no toxicity. Recent studies suggest that cannabis holds promise for the treatment of skin disease [15].

There are over 3.5 million cases of Non- Melanoma Skin Cancer (NMSC) in the US alone, which greatly exceeds the number of all other cancer s combined [16]. NMSC are subdivided into two key types: squamous cell carcinomas and basal cell carcinomas. Basal cell carcinomas comprise 80% of all NMSC cases. This type of cancer develops in cells in the lower epidermis, and occurs most commonly on the face, ears, neck, scalp, shoulders, and back. In the majority of cases basal cell carcinomas grow slowly and rarely metastasize, albeit these tumors can cause significant local disfigurement if untreated or treated inadequately.

Squamous cell carcinoma comprises the 20% of NMSC. Squamous cell carcinomas occur in the upper most layers of the epidermis. These tumors can spread, metastasize and be lethal, whereby of over 700,000 new cases of squamous cell carcinomas diagnosed each year in the United States, over 2,500 will lead to patient's death. NMSC treatments generally involve surgical excision of the tumor together with a margin of normal tissue and, oftentimes in followed by ionizing radiation therapy and chemotherapy.

Although currently available treatments for eczema, psoriasis and NMSC can be relatively effective, they have significant drawbacks and very strong side effects. For example, NMSC surgery can lead to scarring, and radiation and chemotherapy exhibit significant systemic side effects impacting the entire body. Steroids and topical immunomodulators used in psoriasis and eczema treatments (macrolactams, contact sensitizers, immunostimulants) can cause significant side effects, including possible risks of lymphoma and nonmelanoma skin cancer associated with TCIs. The devastating nature psoriasis is emphasized by the extent of the side effects that psoriasis sufferers are willing to endure to attain a remission to a disease.

Therefore, there is a need for further methods of treating skin disorders that are minimally invasive, specific for skin, and easy to use, and have no systemic side effects.

### SUMMARY OF THE INVENTION

It is an object of some aspects of the present invention, to provide improved methods and products for treating, improving and curing skin ailments.

It is an object of some aspects of the present invention to provide compositions for improving wellness in a human or mammalian organism.

It is another object of some aspects of the present invention to provide compositions for preventing or treating diseases or disorders in a human or mammalian organism.

The present invention provides new Cannabis sativa lines and extracts and method of using them as a means to modulate gene expression in skin tissues having an active skin disorder such as psoriasis, eczema, or non-melanoma skin cancer, and others. The disclosure also provides methods of modulating gene expression through the application of novel cannabis extracts to the skin affected by with skin disorders such as non-melanoma skin cancers, psoriasis and atopic dermatitis, and others.

The present invention provides new unique cannabis lines, extracts and methods for skin improvement and healing and reduction in inflammation and skin disease manifestation

The method includes generation of unique lines, whole plant extract preparation, exposing human skin to the extracts in amount sufficient to modulate gene expression in the skin. The modulation of gene expression then results in a reduction of the disease state-associated changes or aspects thereof in the cannabis-treated skin tissues.

In some embodiments of the present invention, improved methods and products are provided for skin treatment.

In other embodiments of the present invention, a method and product is described for improving skin appearance.

In other embodiments of the present invention, a method and product is described for improving skin health.

In additional embodiments for the present invention, new Cannabis sativa lines are provided.

In additional embodiments for the present invention, new extracts from new Cannabis sativa lines are provided.

Accordingly, the present invention provides a method for modulating gene expression (e.g., in skin cells or in skin tissue or in skin tissue disease model) by providing a source of new extracts, exposing skin cells or skin tissue to those in an amount sufficient to modulate gene expression where modulation of gene expression results in a reduction of a disease state in the skin cells or skin tissue.

The present invention further provides freshly prepared extracts of fifteen new C. sativa lines (designated herein lines #4, #6, #8, #13, #14, #18, #5, #31, #39, #49, #69, #114, #166, #267, #273) (FIG. 1) and, using 3D skin psoriasis models (FIG. 2) and EpiDermFT Full Thickness skin models (FIG. 10), identified lines with the best anti-psoriasis properties, based on the expression of genes that are associated with molecular etiology and pathogenesis of skin diseases. These lines included #18, #8, #6, #13, #39, # 166, #49, #31 and #273.

Among the genes down-regulated by extracts of new cannabis lines were members of the Epidermal Differentiation Complex (EDC) genes located on human chromosome 1q21. The EDC comprises 57 genes encoding S100 proteins as well as structural proteins of epidermal cornification. Many of these genes are up-regulated in skin disorders such psoriasis, atopic dermatitis and skin cancers. These genes include members of the Small Proline Rich Region (SPRR), Late Cornified Envelope (LCE), Involucrin (IVL), NICE-1, Cornulin (CRNN) and S100. Other genes down-regulated by cannabis extracts and implicated (up-regulated) in psoriasis are corneodesmosin (CDSN), members of the human β-defensin family (DEFB4 and others), and members of the serine protease inhibitor (serpin) genes and others [5-7]. Deregulation of β-defensins is also implicated in NMSC.

In addition, genes included members of interferon gamma (IFNγ) and interleukin pathways and other pro-inflammatory cytokines upregulation of which has been reposted in psoriasis and eczema. These genes are also implicated in NMSC.

In various embodiments of the present invention, at least one of the genes down-regulated is selected from the group consisting of EDC complex, members of the human β-defensin family (DEFB4 and others), and members of the serine protease inhibitor (serpin) genes, as well as genes of INFγ, interleukin and cytokines/chemokines.

In some embodiments, the gene expression of the skin cells is down-regulated or up-regulated.

In some embodiments, the disease state is a skin disorder. In one specific embodiment, the disease state is psoriasis. In another embodiment, the disease state is atopic dermatitis/eczema. In another embodiment the disease state is an inflammatory skin disease. In another embodiment, the disease state is skin cancer. In some embodiments, the disease state is a non-melanoma skin cancer. In other embodiments, the skin cancer is a basal cell carcinoma or squamous cell carcinoma.

The inventors' current findings suggest potent therapeutic effects against psoriasis, eczema/dermatitis, and NMSC and present a novel and promising natural resource for psoriasis, eczema/dermatitis, and NMSC treatments and modalities.

In various embodiments, at least one of the genes down-regulated is selected from the group consisting of Epidermal Differentiation Complex (EDC) genes, collagen genes, elastin genes, genes involved in modulation of inflammation, oxidative stress, immunity and autoimmunity, cellular matrix, cellular proliferation and apoptosis.

The disclosure provides new unique cannabis lines, extracts dried powders from the extracts, compositions comprising the powders or parts thereof, compounds derived therefrom, pharmaceutical compositions comprising the compound(s), and methods for the treatment of inflammatory skin disorders such as psoriasis, eczema and others. The method includes generation of unique lines, whole plant extract preparation, treating human 3D skin tissues and disease models with extracts in amount sufficient to modulate gene expression in the skin tissues. The modulation of gene expression then results in a reduction of the disease state-associated changes or aspects thereof in the treated skin tissues.

The compositions and dosage forms of the present invention are useful in promoting health and preventing or treating a large number of disorders in human patients and other mammalian subjects.

In additional embodiments of the present invention, compositions and methods are provided for treating and/or preventing skin disorders.

The present invention is directed to compositions and methods for treating disorders, in general, and more particularly, skin diseases and disorders. The compositions of the present invention may be used for improving wellness of a human or mammalian subject. Additionally, the compositions of the present invention may be used to treat any disorder or ailment in a human patient or mammalian subject. Furthermore, the compositions of the present invention may conveniently used in conjunction with a drug to treat any disorder or ailment in a human patient or mammalian subject.

Some embodiments of the present invention provide compounds, compositions and formulations from at least one of hemp and cannabis.

In additional embodiments of the present invention, compositions and methods are provided for treating and/or preventing proliferative disorders.

In additional embodiments of the present invention, compositions and methods are provided for treating and/or preventing cancer.

Some embodiments of the present invention provide compounds, compositions and formulations from at least one of hemp and cannabis.

Some further embodiments of the present invention provide methods for upregulating at least one collagen pathway gene.

Some further embodiments of the present invention provide methods for upregulating expression of at least one retinol metabolism pathway gene.

Some further embodiments of the present invention provide methods for downregulating expression of at least one interleukin gene.

Some further embodiments of the present invention provide methods for downregulating expression of at least one keratinocyte differentiation gene.

Some further embodiments of the present invention provide methods for downregulating expression of at least one keratinocyte proliferation gene.

Some further embodiments of the present invention provide methods for downregulating expression of at least one inflammatory pathway gene.

Some further embodiments of the present invention provide methods for downregulating expression of at least TNF gene.

Some further embodiments of the present invention provide methods for downregulating expression of at least MMP gene.

Some further embodiments of the present invention provide methods for upregulating at least one collagen pathway gene product.

Some further embodiments of the present invention provide methods for upregulating at least one EDC pathway product.

Some further embodiments of the present invention provide methods for downregulating at least one EDC gene product.

Some further embodiments of the present invention provide methods for upregulating at least one LCE pathway product.

Some further embodiments of the present invention provide methods for downregulating at least one LCE gene product.

Some further embodiments of the present invention provide methods for downregulating at least one inflammatory pathway gene product.

Some further embodiments of the present invention provide methods for downregulating at least TNF gene product.

Some further embodiments of the present invention provide methods for downregulating at least MMP gene product.

Some further embodiments of the present invention provide methods for downregulating at least one keratinocyte differentiation gene product.

Some further embodiments of the present invention provide methods for downregulating at least one keratinocyte proliferation gene product.

In particular, there are described methods for preparing compositions, compounds, formulations and extracts for treating a skin disorder or disease, or skin aging, in a human patient.

There is thus provided according to some embodiments of the present invention, a composition, derived from at least one of hemp and cannabis for treating a skin disorder or disease, in a human patient.

A use of a solvent extract from at least one of hemp and cannabis, according to some embodiments of the present invention, is for the manufacture of a pharmaceutical composition for the treatment of a skin disease or disorder.

Some embodiments of the present invention are directed to a method for treating skin in a human patient comprising administering to said patient a pharmaceutically effective amount of the cannabis extract composition as described herein.

Additionally, some further embodiments of the present invention are directed to a method for treating a skin disorder or disease in a human patient comprising administering to said patient the oral dosage form as described herein.

The liquid cannabis extracts of the present invention, and/or dry powders therefrom, are suitable for oral administration, and appear to be well absorbed through the intestine by the blood and thus exhibit the potential to heal a wide range of cancerous organs and inflammatory conditions, such as, but not limited to those mentioned by Chattopadhyay et al. Current Science 87(1) July 2004, 44-53.

According to some embodiments of the present invention, the composition or formulation further comprises at least one solvent or hydrant. In some cases, the hydrant is water, such as double-distilled water. In some cases, it may be at least one organic solvent, such as alcohol.

According to some embodiments of the present invention, the at least one solvent or hydrant is present in the composition or formulation in a concentration of 10-90%, 15-80%, 20-70%, 25-50%, 30-40%, or 10-18% by weight percent.

The solvent or hydrant may further comprise a pH regulator, such as an acid or base. In some embodiments, the base comprises sodium hydroxide.

Suitable products or compositions of the present invention may be in the form of ointments or salves, creams, emulsions, gels, foams, sprays or medicated dressings or bandages, which must be directly applied on the affected zone and must be kept in contact with the skin.

In one or more embodiments, the compositions further comprise up to 10% of water.

In one or more embodiments, the composition is substantially non-aqueous and/or substantially alcohol-free.

In another embodiment, the present invention provides a method for inhibiting a disease in a subject comprising administering a subject a composition of the invention.

In another embodiment, the present invention provides a method for inhibiting a proliferative disease in a subject comprising administering a subject a composition of the present invention.

In another embodiment, the present invention provides a method for inhibiting a disease in a subject comprising orally administering a product of the present invention to the subject.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried cannabis extracts contained therein to be orally administered without drinking.

In one or more embodiments, the composition further comprises a therapeutically effective concentration of one or more active agents.

The composition of the present invention further contains a surface-active agent. Surface-active agents (also termed “surfactants”) include any agent linking oil and water in the composition, in the form of emulsion.

The present invention further provides methods of drug discovery. According to some embodiments, the method includes:
a) combining at least one marijuana cultivar and at least one hemp cultivar to form at least one hybrid line;
b) extracting at least one compound from said at least one hybrid line to form an extract; and
c) testing the extract in vitro to identify a biologically active extract.

The method further includes repeating steps a) to c) on a plurality of extracts to identify the most biologically active extracts.

The method further includes isolating active compounds or components from the biologically active extracts.

The method further comprises treating a patient with a disease or disorder with at least one of the active compounds, components or extracts to cure, alleviate or manage the disease or disorder.

In an embodiment of the present invention, a composition of the present invention includes one or more additional components. Such additional components include but are not limited to anti-static agents, buffering agents, bulking agents, chelating agents, cleansers, colorants, conditioners, diluents, dyes, emollients, fragrances, humectants, permeation enhancers, pH-adjusting agents, preservatives, protectants, skin penetration enhancers, softeners, solubilizers, sunscreens, sun blocking agents, sunless tanning agents, viscosity modifiers and vitamins. As is known to one skilled in the art, in some instances a specific additional component may have more than one activity, function or effect.

### EMBODIMENTS

1. A method for treating mammalian skin, the method comprising:
a) combining at least one marijuana cultivar and at least one hemp cultivar to form at least one Cannabis line;

b) extracting at least one compound from said at least one Cannabis line to form an extract; and

c) treating skin with at least one of said extract and said at least one compound in an effective amount to treat said skin.

2. A method according to embodiment 1, wherein said treating step induces modulation of gene expression in at least one of skin cells and skin tissue; and wherein said modulation of gene said expression results in a reduction of at least one of a skin disease state, an inflammatory state, a skin disorder state, a poor skin appearance state, a skin aging state and combinations thereof.

3. A method according to embodiment 1, wherein said at least one Cannabis line is selected from the group consisting of a marijuana/marijuana hybrid line, hemp/hemp hybrid line and hemp/marijuana hybrid line.

4. A method according to embodiment 3, wherein said at least one line is selected from the group consisting of designated lines #4, #6, #8, #13, #14, #18, #5, #31, #39, #49, #69, #114, #166, #267, #273.

5. A method according to embodiment 1, wherein said extracting step comprises extracting flowers, leaves, roots, stems or any other organ of said at least one Cannabis line.

6. A method according to embodiment 5, wherein said extracting step comprises extracting said at least one compound in at least one organic solvent.

7. A method according to embodiment 6, wherein said extracting step is performed at a temperature in the range of 15-to 60° C. and at a pressure in a range of −0.5 to 1.5 bar and wherein said at least one organic solvent comprises ethyl acetate.

8. A method according to embodiment 2, wherein said modulation of gene expression results in a reduction of a 0.1-3 log₂ fold in at least one of an epidermal differentiation complex (EDC) gene, a keratinocyte differentiation gene and a keratinocyte proliferation gene, an inflammatory marker gene, an inflammation gene and combinations thereof.

9. A method according to embodiment 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT13, KRT15, KRT16P3, KRT 19, KRT23, KRT34, KRT37, KRT7, SPRR2B, SPRR2D, SPRR2F, SPRR2G, SPPP3, SPRR4, CSTA, CNFN, TNC, CHN3, RHCG, FABP3, FABP6, MMP1, MMP10, MMP2, MMP9, DEFB4A, S100A12, S100A7A, S100A8, and S100A9 and combinations thereof.

10. A method according to embodiment 9, wherein said at least one Cannabis line comprises line #18.

11. A method according to embodiment 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT10, SERPINB12, BTC ANDS100A7A and combinations thereof.

12. A method according to embodiment 11, wherein said at least one Cannabis line comprises line #14.

13. A method according to embodiment 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT 19, KRT37, KRT7, SPRR2A, SPRR2B , SPRR4, CNFN, RHCG, MMP10, DEFB4A, S100A7A, CCL8, IL23A and IL32 and combinations thereof.

14. A method according to embodiment 13, wherein said at least one Cannabis line comprises line #13.

15. A method according to embodiment 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT15, KRT 19, KRT7, KRT8, SERPIN F1, MMP10, MMP14, MMP2 MMP9, DEFB4A, S100A7A, CXCL1, CXCL14, CXCL15, CXCL6, and IL32 and combinations thereof.

16. A method according to embodiment 15, wherein said at least one Cannabis line comprises line #8.

17. A method according to embodiment 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT19, KRT42P, KRT17, DCN, CNFN, FABP3, MMP10, MMP17, MMP2, MMP3, MMP9, DEFB4A, S100A12, S100A7, S100A7A, S100A8, S100A9, IL13RA1, IL13RA2, IL1R1, IL1R2 IL1RL1, IL24, IL2RG, IL32, GJB2, CXCL1, CXCL5, CXCL6, CXCL8 and combinations thereof.

18. A method according to embodiment 17, wherein said at least one Cannabis line comprises line #6.

19. A method according to embodiment 2, wherein said modulation of gene expression results in at least one of an increase or a reduction in a 0.1-3 log₂ fold in at least one of an epidermal differentiation complex (EDC) gene, a keratinocyte differentiation gene and a keratinocyte proliferation gene, an inflammatory marker gene, an inflammation gene and combinations thereof.

20. A method according to embodiment 19, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT73, KRT79, MMP10, MMP11, MMP14, MMP19, MMP2, MMP3, MMP8, TIMP1, TIMP2 and S100A7A and combinations thereof.

21. A method according to embodiment 20, wherein said at least one Cannabis line comprises line #4.

22. A method according to embodiment 2, wherein said modulation of gene expression results in at least one of a reduction in a 0.1-3 log₂ fold in at least one of a Late Cornified Envelope (LCE) gene, a epidermal differentiation complex (EDC) gene, a keratinocyte differentiation gene and a keratinocyte proliferation gene, an inflammatory marker gene, an inflammation gene and combinations thereof.

23. A method according to embodiment 22, wherein said Late Cornified Envelope (LCE) gene is selected from the group consisting of LCE1A, LCE1B, LCE1C, LCE1D, LCE1E, LCE1F, LCE3A, LCE5A, LCE6A and combinations thereof.

24. A method according to embodiment 23, wherein said at least one Cannabis line comprises line #13.

25. A method according to embodiment 2 where said modulation of gene expression in at least one of a reduction in a 0.1-3 log₂ fold in at least one of a Late Cornified Envelope (LCE) gene, a epidermal differentiation complex (EDC) gene, a keratinocyte differentiation gene and a keratinocyte proliferation gene, an inflammatory marker gene, an inflammation gene and combinations thereof.

26. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: CSTA, CSTB, DEFB1, S100A10, S100A11,S100A12,S100A14,S100A16, S100A7, S100A9, SERPINA12, SERPINB1, SERPINB12, SERPINB13, SERPINB3, SERPINB4, SERPINB 5, SPRR1A, SPRR1B, MPP7, KRT1, KRT10, KRT13, KRT14, KRT15, KRT16, KRT16P6, KRT2, KRT23, KRT5, KRT6A, KRT6B, KRT6C, KRT75, KRTDAP, TNFRSF10A, TNFSF10, IL1 8, IL1RAP, IL20RA, IL20RB, IL33, CXCL12, CXCL14

27. A method according to embodiment 26, wherein said at least one Cannabis line comprises line #39.

28. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: CXCL14, IL1RAP, IL20RB, IL33, TNFSF10, KRT1, KRT10, KRT14, KRT15, KRT16P6, KRTS, KRT6B, KRT6C, MPP7, S100A9, SERPINB3, SERPINB4, SERPINB5, SPRR1A, SPRR1B.

29. A method according to embodiment 29, wherein said at least one Cannabis line comprises line 190166.

30. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: CSTA, CXCL14, KRT1, KRT10, KRT6C, MPP7, S100A11, S100A9, SERPINB3, SERPINB4, SERPINB5, SPRR1B, IL33.

31. A method according to embodiment 29, wherein said at least one Cannabis line comprises line #273.

32. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: CXCL1, CSTA, S100A11, SERPINB3, SERPINB5, SPRR1B

33. A method according to embodiment 32, wherein said at least one Cannabis line comprises line #5.

34. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: CSTA, KRT6C, S100A11, S100A9, SERPINB13, SERPINB3, SERPINB 4, SERPINB5, SPRR1A, SPRR1B

35. A method according to embodiment 34, wherein said at least one Cannabis line comprises line #31.

36. A method according to embodiment 25, wherein said keratinocyte differentiation gene and a keratinocyte proliferation gene, and tissue remodelling gene is selected from the group consisting of: KRT6C, MMP11

37. A method according to embodiment 36, wherein said at least one Cannabis line comprises line #69.

38. A method according to embodiment 25, wherein said Late Cornified Envelope (LCE) gene, a epidermal differentiation complex (EDC) gene, a keratinocyte differentiation gene and a keratinocyte proliferation gene, an inflammatory marker gene, an inflammation gene is selected from the group consisting of: SERPINB5, KRT1, KRT10, KRT14, KRT15, KRT16, KRT2, KRTS, KRT6B, KRT6C

39. A method according to embodiment 38, wherein said at least one Cannabis line comprises line #49.

40. A method according to embodiment 25, wherein said keratinocyte differentiation gene and a keratinocyte proliferation gene, and inflammation gene is selected from the group consisting of: CXCL14, KRT14

41. A method according to embodiment 40, wherein said at least one Cannabis line comprises line #114.

42. A method according to embodiment 25, wherein said Late Cornified Envelope (LCE) gene, and keratinocyte differentiation gene and a keratinocyte proliferation gene, and inflammation gene is selected from the group consisting of: SERPINB4, IL33, KRT6C

43. A method according to embodiment 42, wherein said at least one Cannabis line comprises line #267.

44. A method according to embodiment 1, wherein said at least one compound is provided in a concentration in a range of 0.0001-0.05 μg/μl, 0.001-0.05 μg/μl, 0.001-0.005 μg/μl, 0.003-0.03 μg/μl or 0.007-0.015 μg/μl.

45. A method according to embodiment 1, wherein said at least one compound is provided in a solvent extract and said solvent extract exhibits skin healing properties.

46. A method according to embodiment 6, wherein said solvent extract is at least 2-20, 3-15, 4-12, 5-10 or 6-9 times as effective as at least one of THC (tetrahydrocannabinol) and CBD (cannabidiol), administered at the same concentration in treating said disease.

47. The method of embodiment 2, wherein the disease state is skin cancer.

48. The method of embodiment 48, wherein the skin cancer is non-melanoma skin cancer.

49. The method of embodiment 49, wherein the non-melanoma skin cancer is a squamous cell carcinoma or basal cell carcinoma.

50. The method of embodiment 2, wherein the disease state is an inflammatory skin disease.

51. The method of embodiment 50, wherein the disease state is selected from the group consisting of a skin insult, a skin disorder, a skin disease, psoriasis, atopic dermatitis, contact dermatitis, or UV-induced inflammation, environmental factor-induced inflammation and combinations thereof.

52. The method of embodiment 47, wherein the skin cancer is non-melanoma skin cancer.

53. A method according to embodiment 1, wherein said Cannabis line is a Cannabis sativa line.

54. An organic extract of at least one plant line, said at least one plant line formed from combining at least one of:
a) at least one marijuana or hemp cultivar; and
b) at least one other marijuana or hemp cultivar,

wherein said organic extract comprises at least one compound suitable for treating a mammalian skin disease or disorder.

55. An organic extract according to embodiment 54, wherein said at least one plant line comprises a Cannabis sativa line.

56. An organic extract according to embodiment 54, wherein said mammalian skin disease or disorder is selected from the group consisting of skin cancer, non-melanoma skin cancer, squamous cell carcinoma, basal cell carcinoma, cancer, an inflammatory skin disease, psoriasis, atopic dermatitis, contact dermatitis, a UV-induced disorder, a burn, a cut, a scar, a skin insult, or other environmental factor-induced inflammation or disease.

57. An organic extract according to embodiment 54, wherein said extract is effective against chemo-resistant cancer cells and is suitable to overcome chemo-resistance.

58. An organic extract according to embodiment 54, wherein said extract potentiates effects of cytotoxic chemotherapy and is an effective and safe adjuvant modality.

59. An organic extract according to embodiment 54, wherein said organic extract is at least 2-20, 3-15, 4-12, 5-10 or 6-9 times as effective as at least one of THC and CBD, administered at the same concentration in treating said disease.

60. A combination therapy, isolated from an organic extract of at least one hybrid line, said at least one hybrid line formed from combining at least one of:
a) at least one marijuana cultivar; and
b) at least one hemp cultivar; and

wherein said organic extract comprises a plurality of compounds suitable for treating a mammalian skin disease or disorder.

61. A combination therapy according to embodiment 60, wherein said mammalian skin disease or disorder is selected from the group consisting of skin cancer, non-melanoma skin cancer, squamous cell carcinoma, basal cell carcinoma, cancer, an inflammatory skin disease, psoriasis, atopic dermatitis, contact dermatitis, a UV-induced disorder, a burn, a cut, a scar, a skin insult, or other environmental factor-induced inflammation.

62. A line of Cannabis sativa formed by combining at least one marijuana cultivar and at least one hemp cultivar, said line to be deposited at a publicly available culture collection, designated herein #4, #6, #8, #13, #14, #18, #5, #31, #39, #49, #69, #114, #166, #267, #273.

63. A line according to embodiment 62, wherein the extracts is extract #18.

64. A line according to embodiment 62, wherein the extracts is extract #14.

65. A line according to embodiment 62, wherein the extracts is extract #13.

66. A line according to embodiment 62, wherein the extracts is extract #8.

67. A line according to embodiment 62, wherein the extracts is extract #6

68. A line according to embodiment 62, wherein the extracts is extract #4.

69. A line according to embodiment 62, wherein the extracts is extract #5.

70. A line according to embodiment 62, wherein the extracts is extract #31.

71. A line according to embodiment 62, wherein the extracts is extract #39.

72. A line according to embodiment 62, wherein the extracts is extract #49.

73. A line according to embodiment 62, wherein the extracts is extract #69.

74. A line according to embodiment 62, wherein the extracts is extract #81.

75. A line according to embodiment 62, wherein the extracts is extract #114.

76. A line according to embodiment 62, wherein the extracts is extract #166.

77. A line according to embodiment 62, wherein the extracts is extract #267.

78. A line according to embodiment 62, wherein the extracts is extract #273.

The present invention will be more fully understood from the following detailed description of the preferred embodiments thereof, taken together with the drawings.

### BRIEF DESCRIPTION OF THE DRAWINGS

The invention will now be described in connection with certain preferred embodiments with reference to the following illustrative figures so that it may be more fully understood.

With specific reference now to the figures in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the context of this application, the word “Figure” is abbreviated to Fig. In the drawings:

FIGS. 1A-1O show the results of high performance liquid chromatography (HPLC) profiles of tested lines, in accordance with some embodiments of the present invention;

FIG. 2A shows psoriasis tissue models and experimental set-up, in accordance with some embodiments of the present invention. H&E stained histological (formalin fixed) cross-sections of (top) in vitro reconstructed psoriasis tissue model (SOR-300-FT) containing normal human keratinocytes and psoriatic fibroblasts, and (bottom) psoriasis-involved skin explants. Image courtesy of MatTek Corporation. FIG. 2B. Scheme of the experiment—extracts were applied on psoriasis tissue models and gene expression changes were analysed;

FIG. 3A shows the effect of extract #18: Differentially expressed Epidermal Differentiation Complex (EDC) genes involved in keratinization and proliferation; FIG. 3B selected other genes associated with non-melanoma skin cancer or inflammatory skin diseases. Graphs show log₂ fold changes for differentially-expressed genes, in accordance with some embodiments of the present invention;

FIG. 4 shows the effect of extract #14: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 5 shows the effect of extract #13: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 6 shows the effect of extract #8: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 7 shows the effect of extract #6: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 8 shows the effect of extract #4: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 9 shows the expression of LCE genes in PSOR skin upon treatment with extracts. Graphs show log₂ fold changes for differentially-expressed genes. No LCE genes were changed by extract #13, in accordance with some embodiments of the present invention;

FIG. 10A shows the EpiDermFt tissues and experimental set-up. EpiDermFT (Mattek) has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. The model is widely used and accepted for platform for studying effects of various topical applications on skin [17]. FIG. 10B. Tissue insert in a well with medium. FIG. 10C. Scheme of experiments. To analyze the direct effects of extracts on skin, extracts were dissolved in coconut oil and applied directly to the skin model surface, in accordance with some embodiments of the present invention.

FIG. 11 shows the effect of extracts #49, #69, #114, #267, #5, #31, #273: Differentially expressed Epidermal Differentiation Complex (EDC) genes involved in keratinization and proliferation and selected other genes associated with non-melanoma skin cancer or inflammatory skin diseases. Graphs show logₑ fold changes for differentially-expressed genes, in accordance with some embodiments of the present invention;

FIG. 12 shows the effect of extract #166: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention;

FIG. 13 shows the effect of extract #39: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes, in accordance with some embodiments of the present invention.

FIG. 14 shows a schematic summary of a methodology for finding active components for treating skin diseases, in accordance with some embodiments of the present invention.

In all the figures similar reference numerals identify similar parts.

### DETAILED DESCRIPTION OF THE EMBODIMENTS

In the detailed description, numerous specific details are set forth in order to provide a thorough understanding of the invention. However, it will be understood by those skilled in the art that these are specific embodiments and that the present invention may be practiced also in different ways that embody the characterizing features of the invention as described and claimed herein.

### Materials and Methods

### Plant Crude Extract Preparation

Solvent used: Ethyl acetate ACS grade from Fisher cat# E145-4 (99.9% pure) Extract Preparation: 3 g of the powdered plant tissue were weighed using an analytical balance Plant material was placed inside a 250 mL Erlenmeyer flask (clean). 100 mL of Ethyl Acetate was poured into the flask containing the plant material. The flasks were then wrapped with tin foil and shaken continuously (120 rpm) in an incubator @21° C. overnight and in the dark.

After overnight solvent extraction the extracts were filtered through cotton into a 100 mL round bottom flask. The extracts were concentrated to around 2-3 mL using a rotary vacuum evaporator. The extracts were then transferred to a tared 3 dram vial (cat# 60975L Kimble obtained from Fisher Scientific). The leftover solvent was evaporated to dryness in an oven overnight @50 C to eliminate the solvent completely. Mass of each extract was recoded.

### Bioassay Preparation

Preparation of 60 mg/mL Stocks.

The stocks were prepared weighing a 3-6 mg of crude extract into a micro centrifuge tube. The crude extract was dissolved in DMSO (Dimethyl sulfoxide anhydrous from Life technologies cat # D12345) to reach 60 mg/mL final concentration and stored at −20C.

Preparation of Crude Extracts for Bioassay.

Appropriate cell culture media (in these experiments RPMI+10% FBS or EMEM+10% FBS) was used to dilute the 60 mg/mL stock. The stocks are allowed to thaw then added to the cell culture media, mixed thoroughly to ensure they are in solution and filtered through a 0.22 um syringe filter. These filtrates were ready to be applied to cells and tested.

### Models

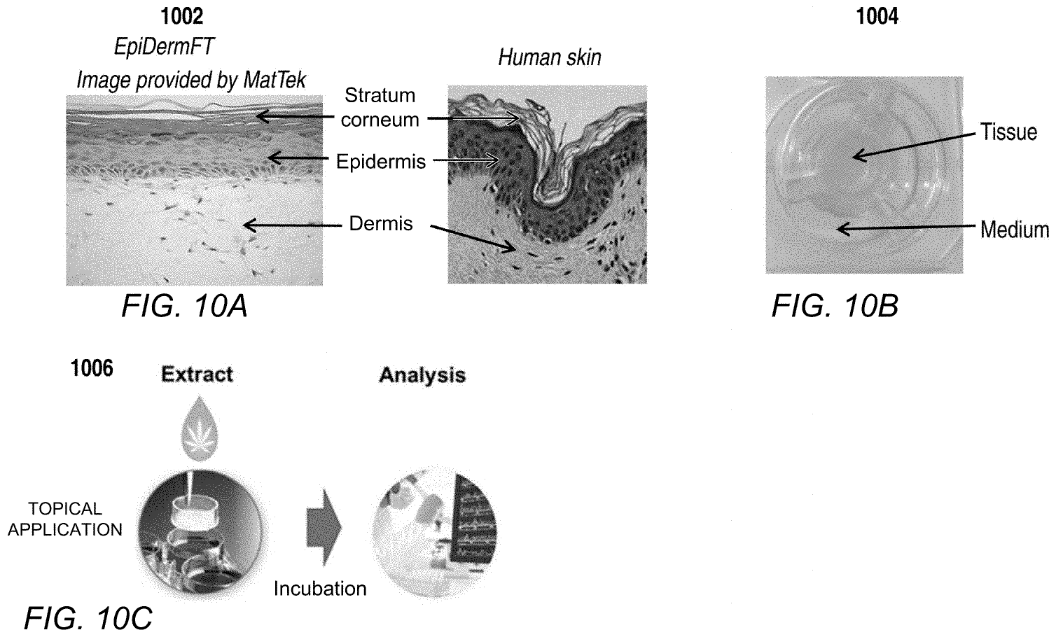

EpiDermFT Full Thickness Skin Model: This model, developed by MatTek Corporation, consists of normal, human-derived epidermal keratinocytes and normal, human-derived dermal fibroblasts, which have been cultured to form a multilayered, highly differentiated model of the human dermis and epidermis (FIG. 10).

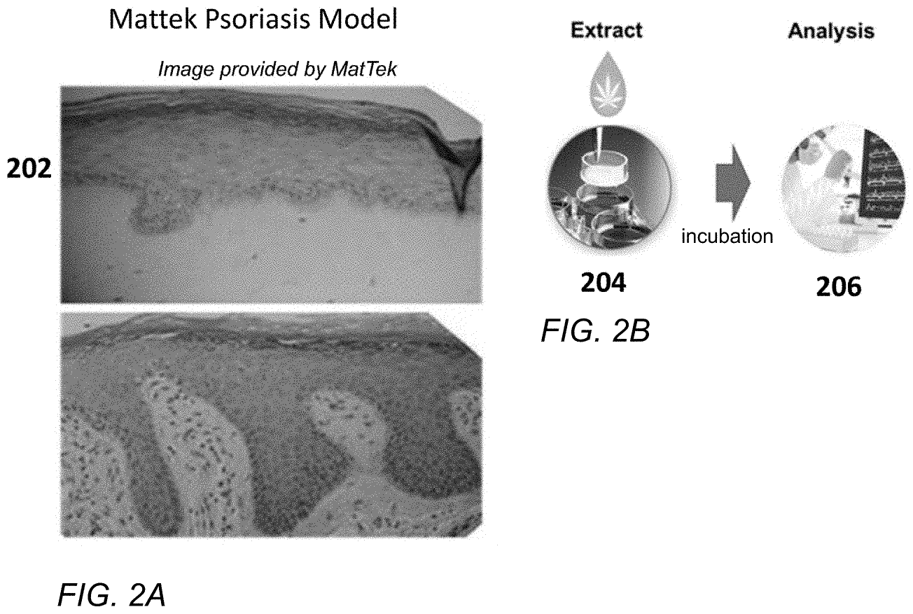

SOR, a Psoriasis Skin Model: Psoriasis is a chronic inflammatory skin disease characterized by epidermal hyperplasia (acanthosis) with abnormal differentiation of epidermal keratinocytes and accumulation of polymorphonuclear leukocytes, altered dermal angiogenesis, infiltration of activated T cells, as well as increased inflammatory cytokine levels. SOR-300-FT is a novel organotypic psoriasis tissue model developed by MatTek Corporation. The SOR-300-FT tissues are cultured using normal human epidermal keratinocytes and psoriatic fibroblasts harvested from psoriasis lesions. They are characterized by increased basal cell proliferation, expression of psoriasis-specific markers and cytokines related to psoriasis. Morphologically, SOR-300-FT tissues highly resemble psoriatic human tissues (FIG. 2).

### Gene Expression Profiling

Three tissues per group were used for the analysis of gene expression profiles. RNA was extracted from tissues using TRIzol® Reagent (Invitrogen, Carlsbad, Calif.), further purified using an RNAesy kit (Qiagen), and quantified using Nanodrop2000c (ThermoScientific). Afterwards, RNA integrity and concentration were established using 2100 BioAnalyzer (Agilent). Sequencing libraries were prepared using Illumina's TruSeq RNA library preparation kits, and global gene expression profiles were determined using the Next 500 Illumina deep-sequencing platform at the University of Lethbridge Facility. Statistical comparisons between the control and treatment groups were performed using the DESeq Bioconductor package (version 1.8.3) and the baySeq Bioconductor package (version 1.10.0). Clustering of the samples was assessed with multidimensional scaling (MDS) plots built using the plotMDS function from the edgeR Bioconductor package. Features with a false discovery rate (FDR) <0.1 (10% false positive rate) were considered differentially expressed between conditions.

The functional annotations of differentially expressed genes were performed using David, GO (Gene Ontology) Elite, and GO-TermFinder. Pathways were visualized using Pathview/KEGG and DAVID bioinformatics platforms DAVID Bioinformatics Resources 6.7 KEGG Pathway platforms.

### Results

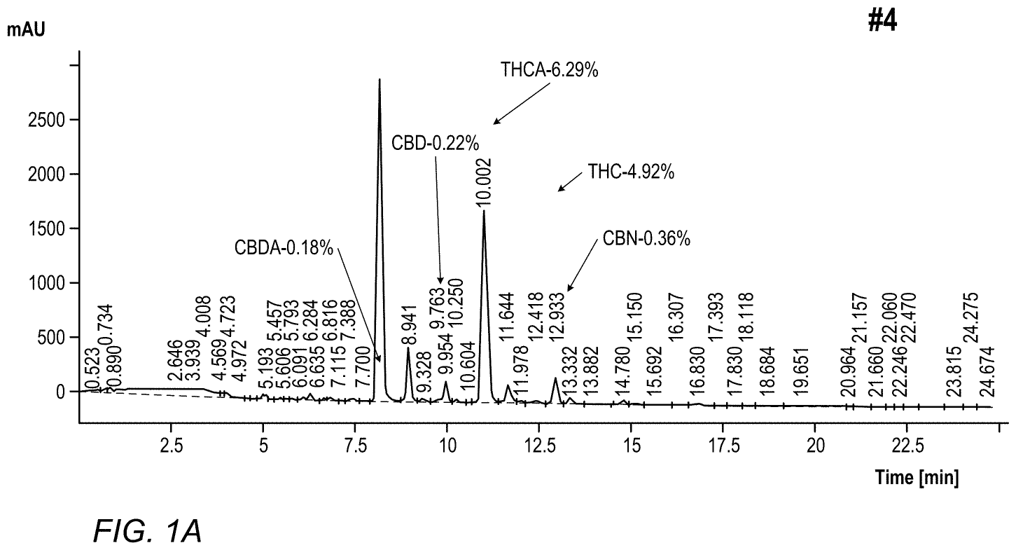

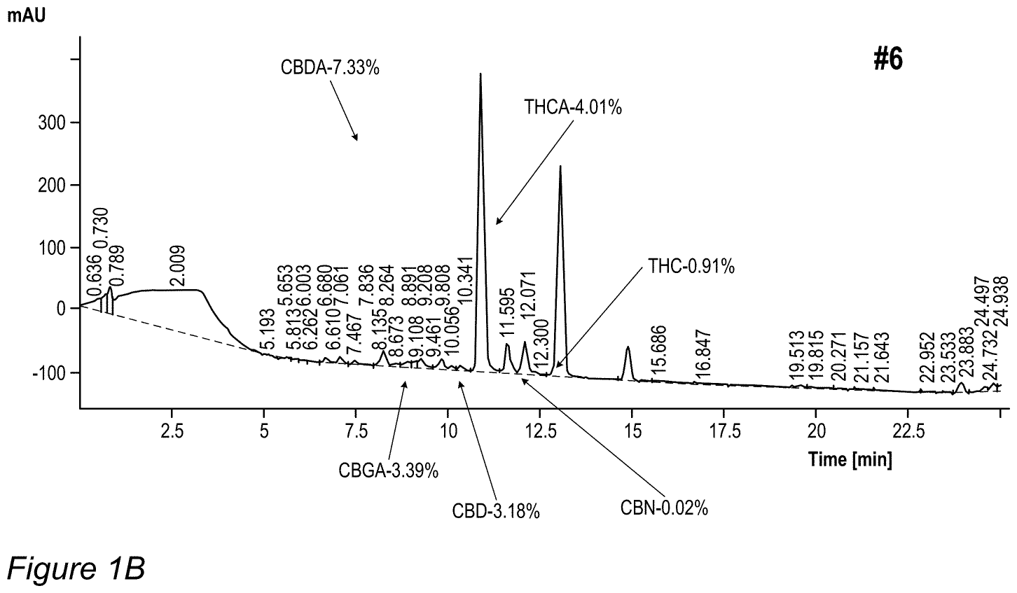

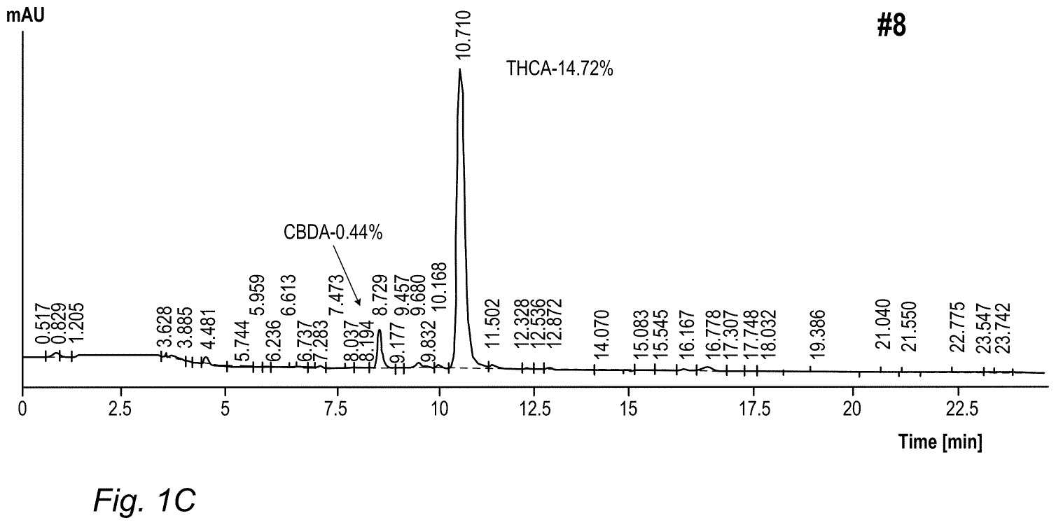

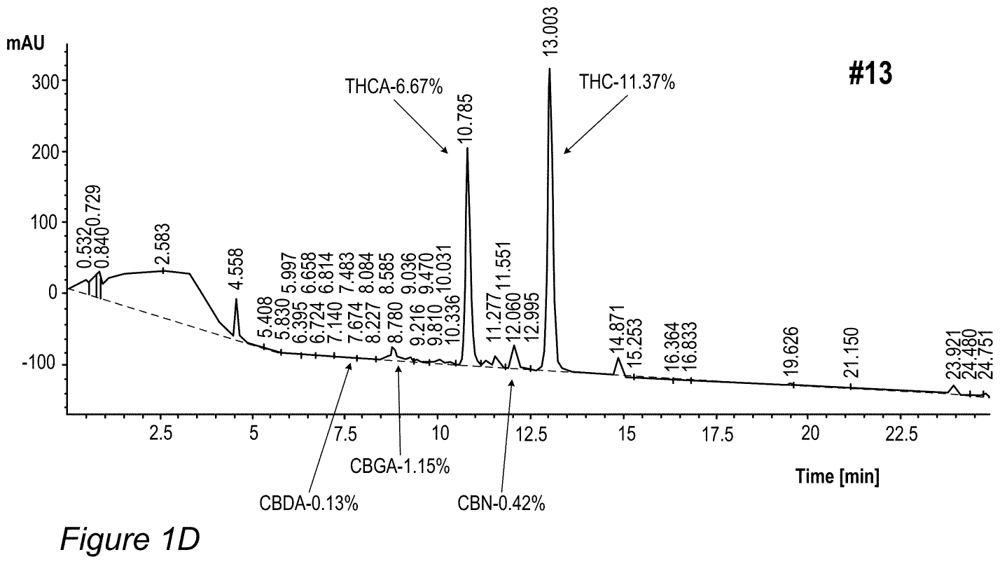

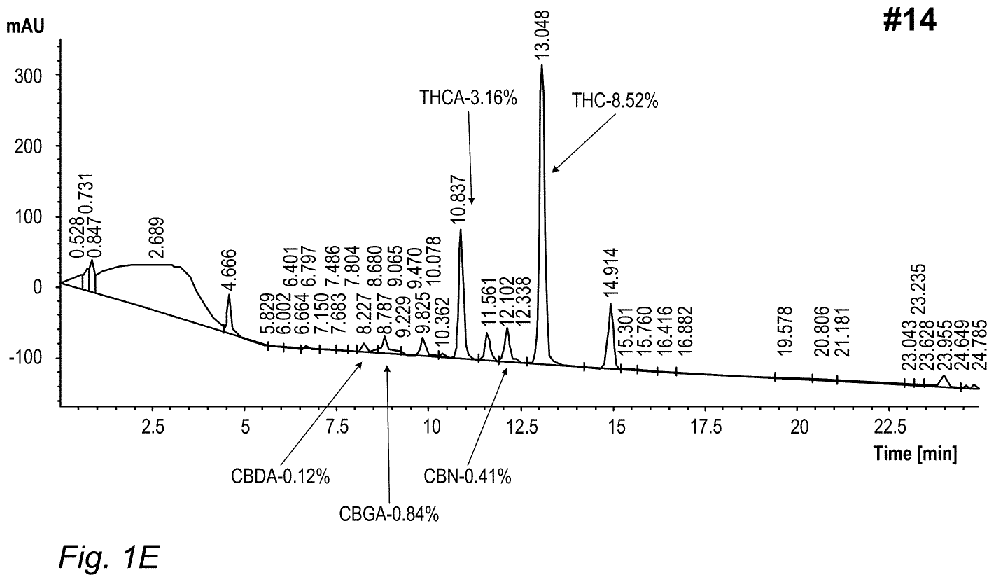

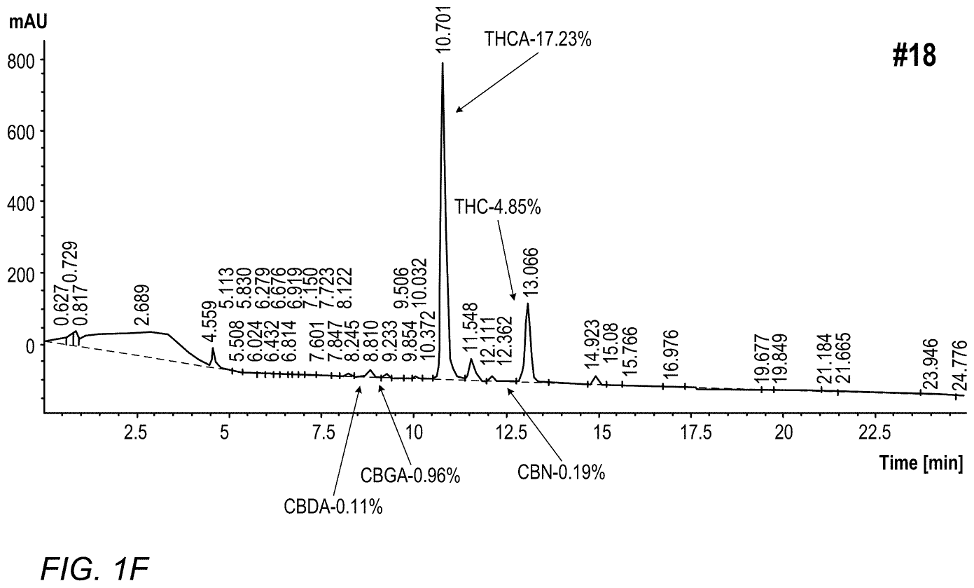

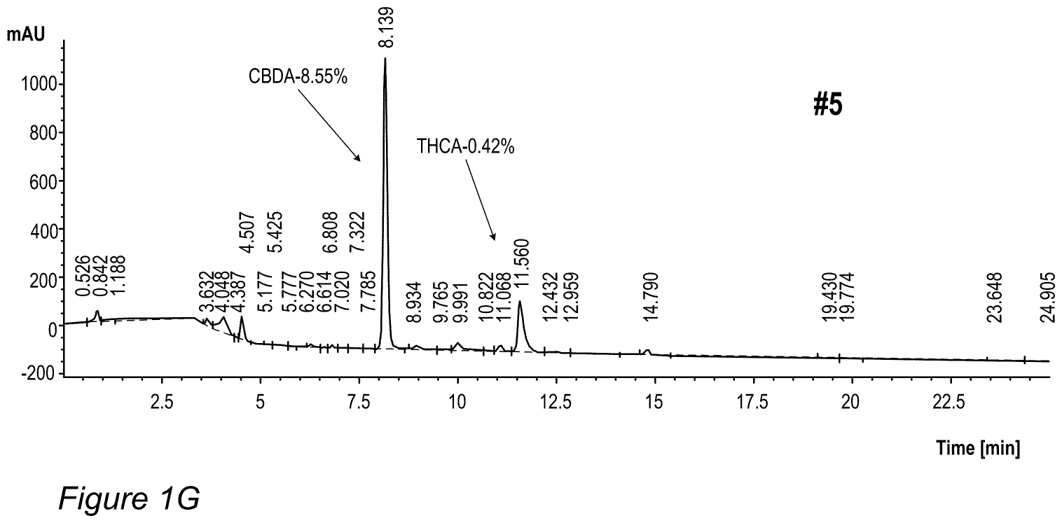

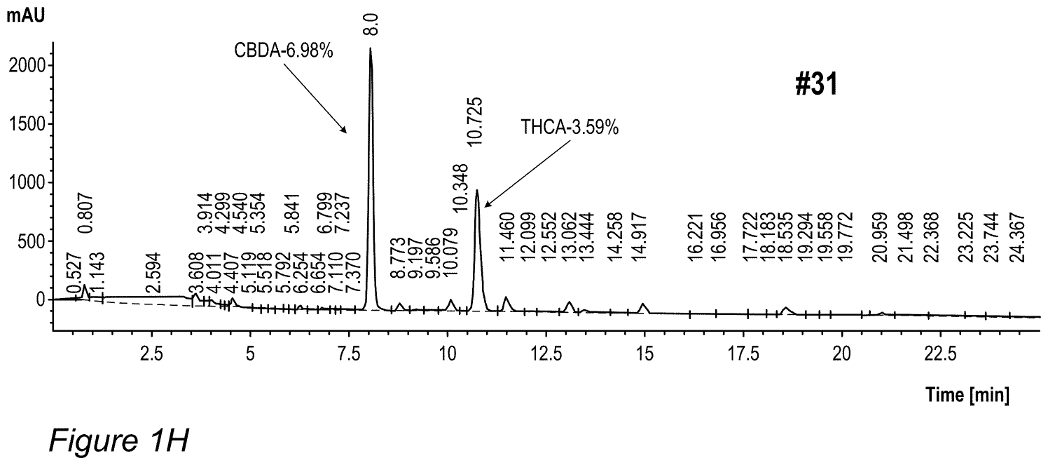

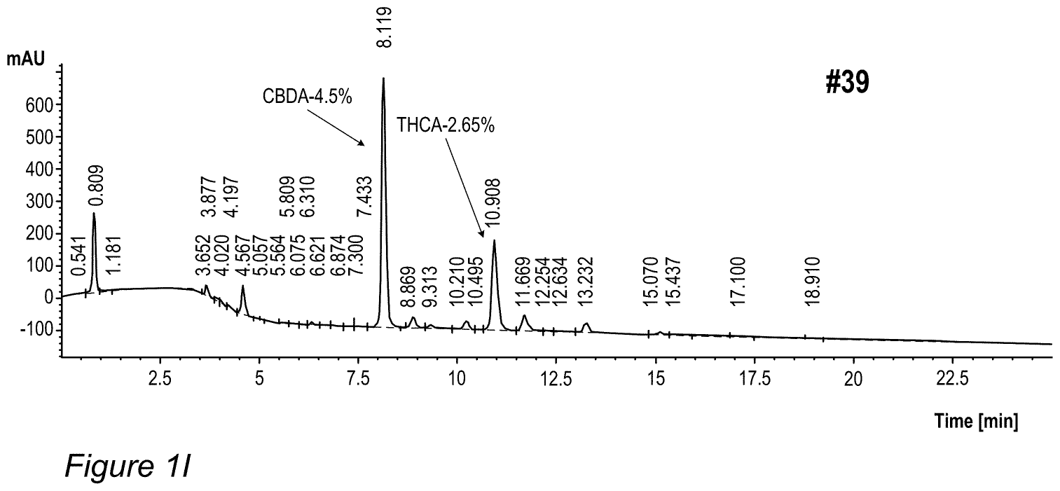

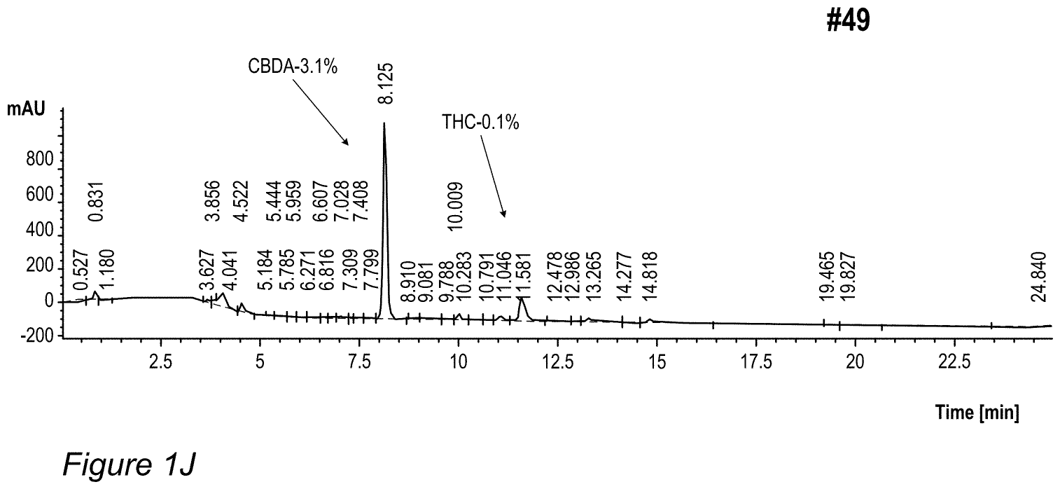

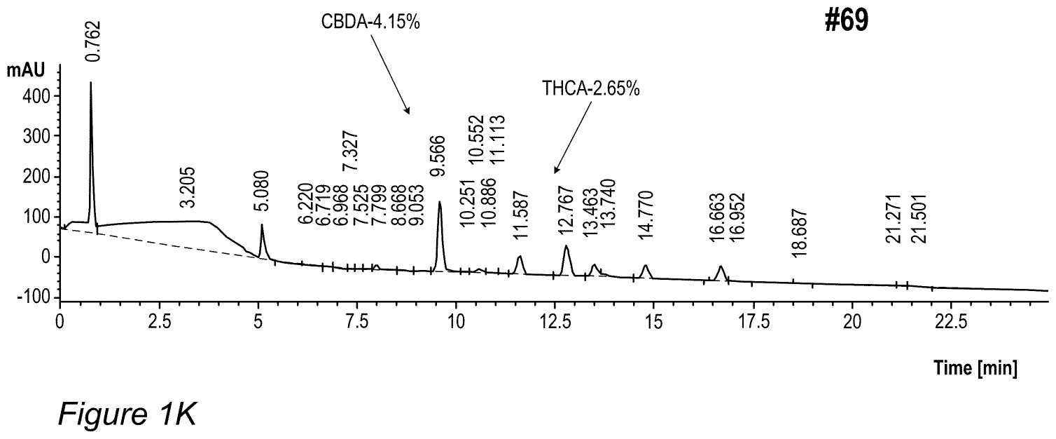

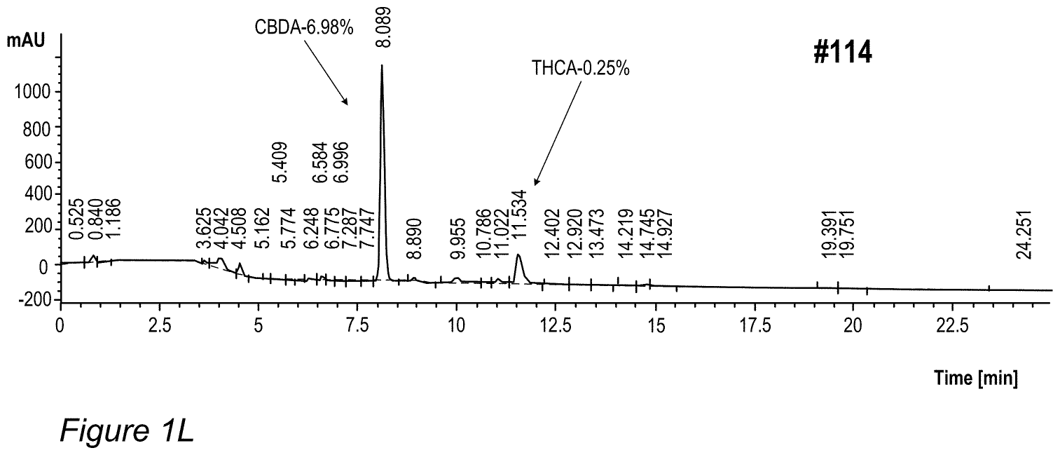

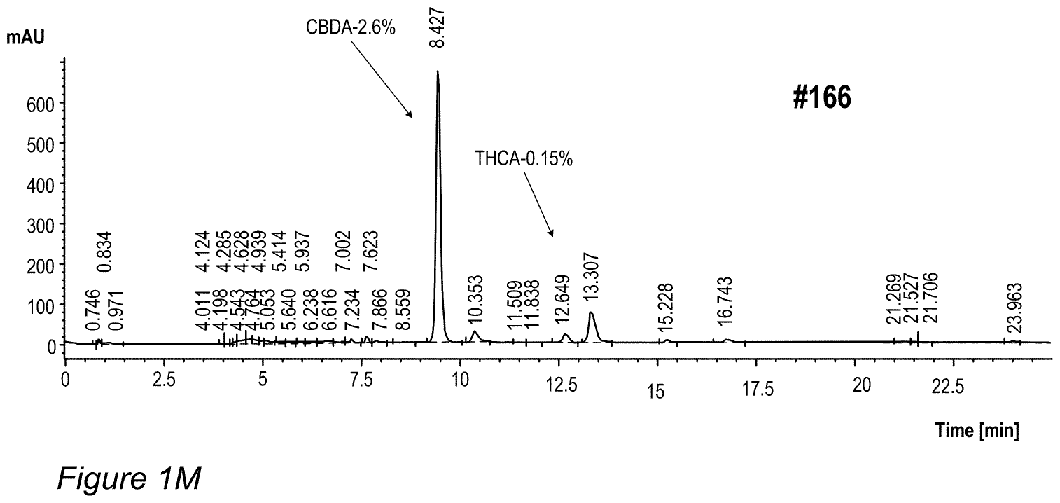

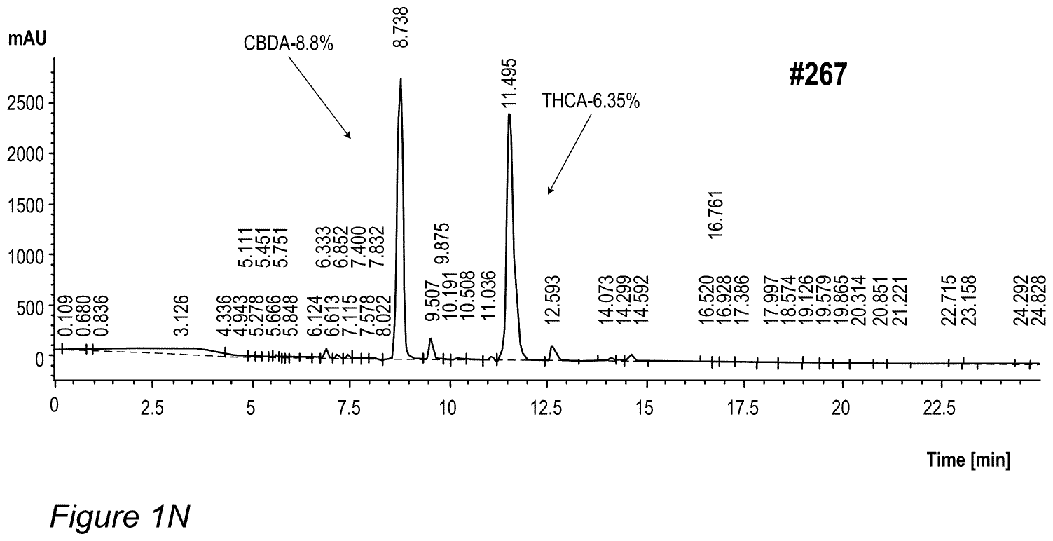

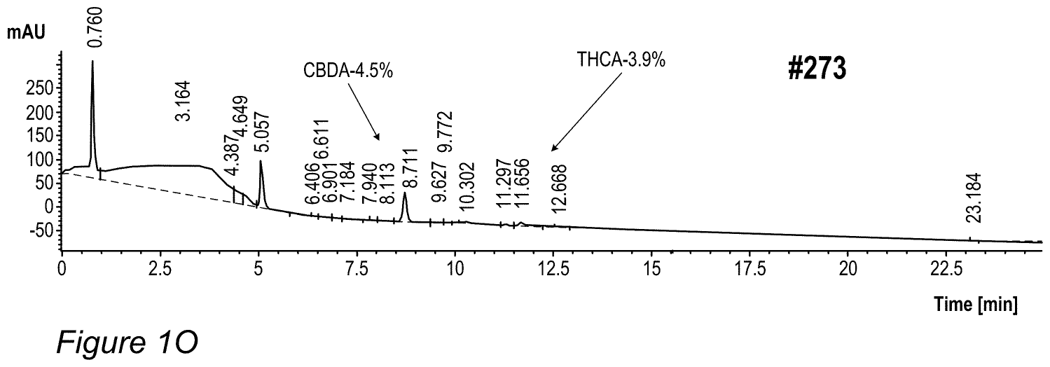

FIGS. 1A-1O show high performance liquid chromatography (HPLC) profiles of tested lines, in accordance with some embodiments of the present invention:

| FIG. 1 panel | LINES (#) | THC | CBD |
|---|---|---|---|
| FIG. 1A | 4 | 10.44 | 0.38 |
| FIG. 1B | 6 | 4.43 | 9.61 |
| FIG. 1C | 8 | 14.72 | 0.41 |
| FIG. 1D | 13 | 17.22 | 0.21 |
| FIG. 1E | 14 | 11.3 | 0.42 |
| FIG. 1F | 18 | 19.96 | 0.1 |
| FIG. 1G | 5 | 0.27 | 8.46 |
| FIG. 1H | 31 | 3.54 | 6.90 |
| FIG. 1I | 39 | 2.6 | 4.4 |
| FIG. 1J | 49 | 0.1 | 3.05 |
| FIG. 1K | 69 | 2.56 | 4.1 |
| FIG. 1L | 114 | 0.18 | 6.92 |
| FIG. 1M | 166 | 0.1 | 2.5 |
| FIG. 1N | 267 | 6.8 | 8.2 |
| FIG. 1O | 273 | 3.8 | 4.4 |

FIG. 2A. Psoriasis tissue models and experimental set-up. H&E stained histological (formalin fixed) cross-sections of (top) in vitro reconstructed psoriasis tissue model (SOR-300-FT) containing normal human keratinocytes and psoriatic fibroblasts, and (bottom) psoriasis-involved skin explants. Image courtesy of MatTek Corporation. FIG. 2B. Scheme of the experiment—extracts were applied on psoriasis tissue models and gene expression changes were analysed.

In tissue preparation step 202 psoriasis tissue model (SOR-300-FT) was used. This tissue model is similar to actual psoriasis plaque from a patient. 3D tissues were inserted in a well with medium. Tissues were equilibrated in EFT-400 for 24 h (overnight) then culture media EFT-400 was replaced and incubated for another 24 h.

In the extract application step 204, extracts were dissolved in coconut oil and applied directly to the 3D tissue surface for 24 h upon which the samples were taken for the analysis.

In the bioinformatics analysis step 206, samples were used for mRNA sequencing and analysis of changes in biological pathways associated with psoriasis.

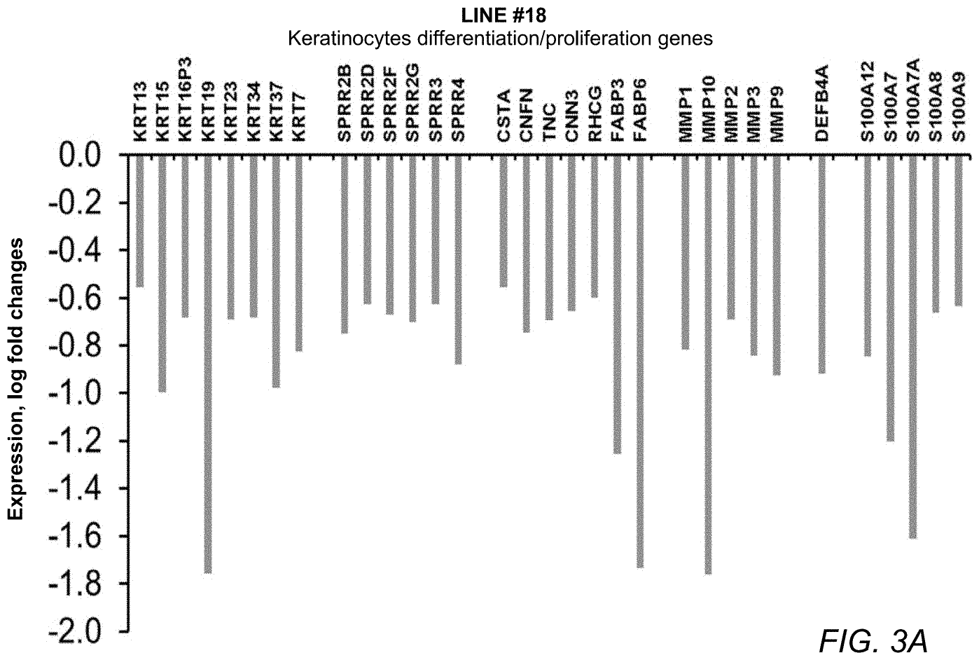

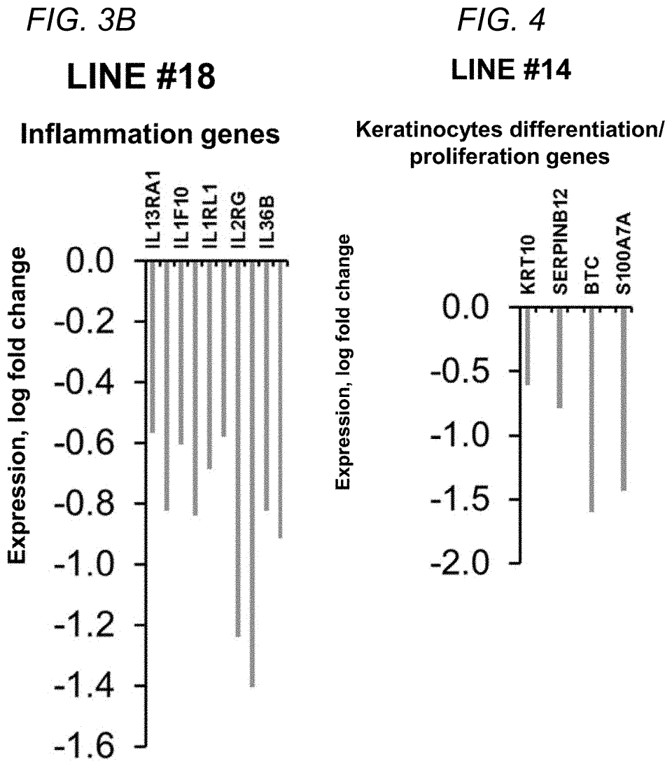

FIG. 3A shows the effect of extract #18: Differentially expressed Epidermal Differentiation Complex (EDC) genes involved in keratinization and proliferation: FIG. 3B shows selected other genes associated with non-melanoma skin cancer or inflammatory skin diseases. Graphs show log₂ fold changes for differentially-expressed genes.

FIG. 4 shows the effect of extract #14: Differentially expressed Epidermal Differentiation Complex (EDC) genes involved in keratinization and proliferation and selected other genes associated with non-melanoma skin cancer or inflammatory skin diseases. Graphs show logₑ fold changes for differentially-expressed genes.

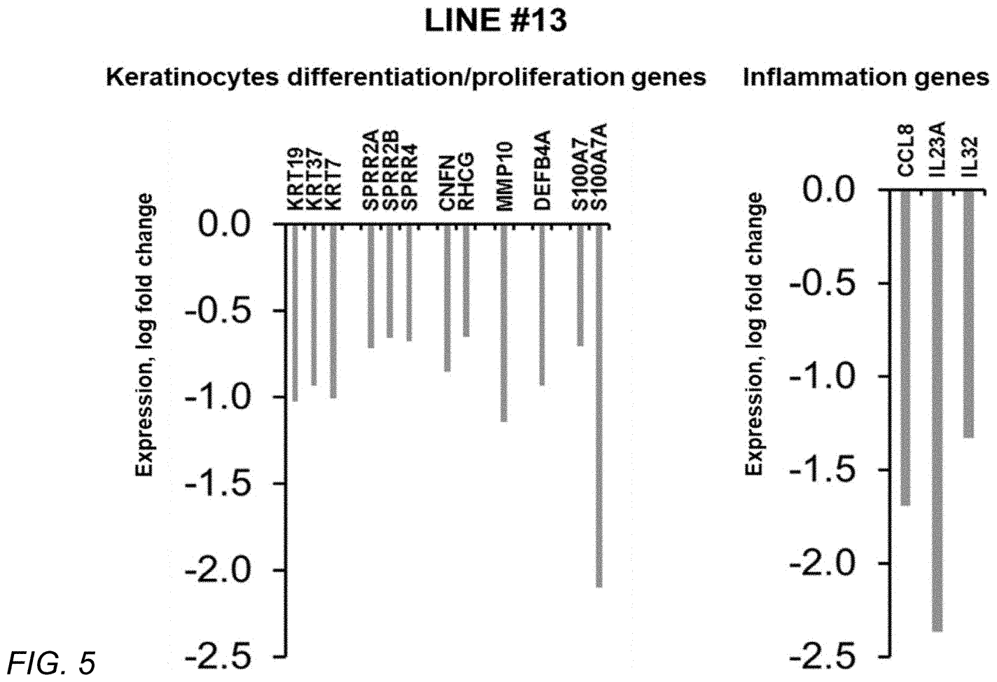

FIG. 5 shows the effect of extract #13: Differentially expressed Epidermal Differentiation Complex (EDC) genes involved in keratinization and proliferation and selected other genes associated with non-melanoma skin cancer or inflammatory skin diseases. Graphs show logₑ fold changes for differentially-expressed genes.

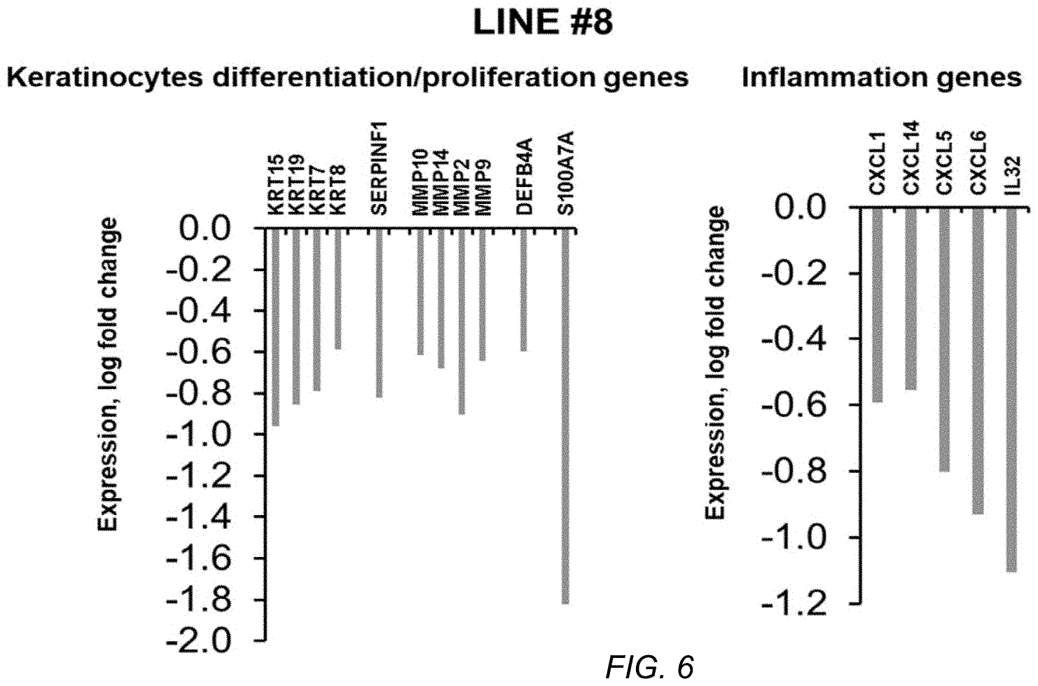

FIG. 6 shows the effect of extract #8: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

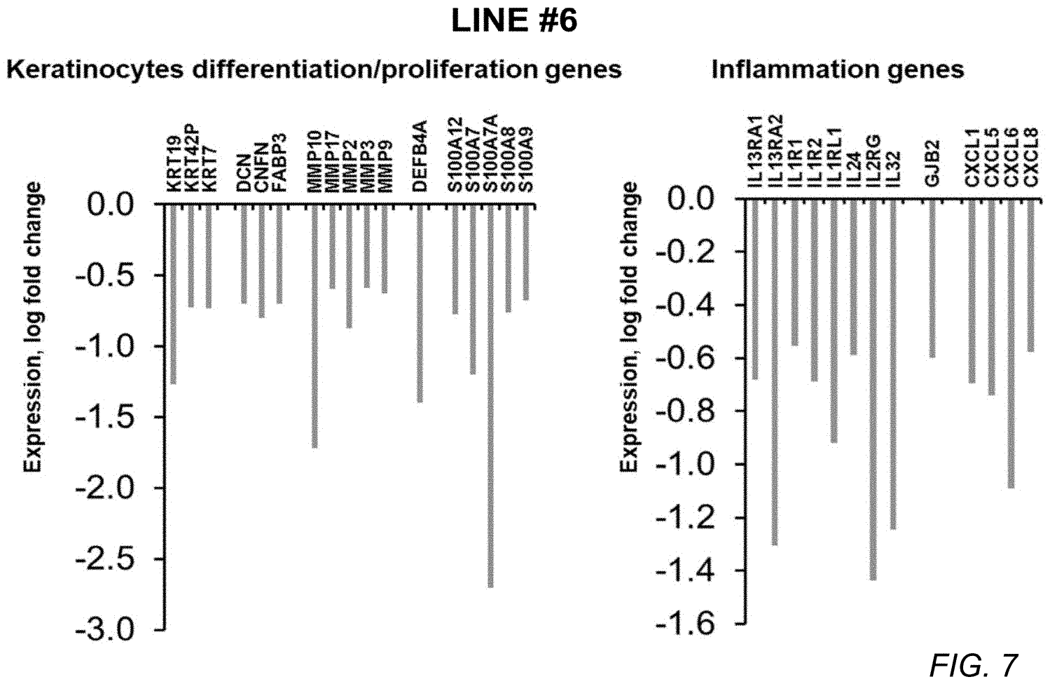

FIG. 7 shows the effect of extract #6: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

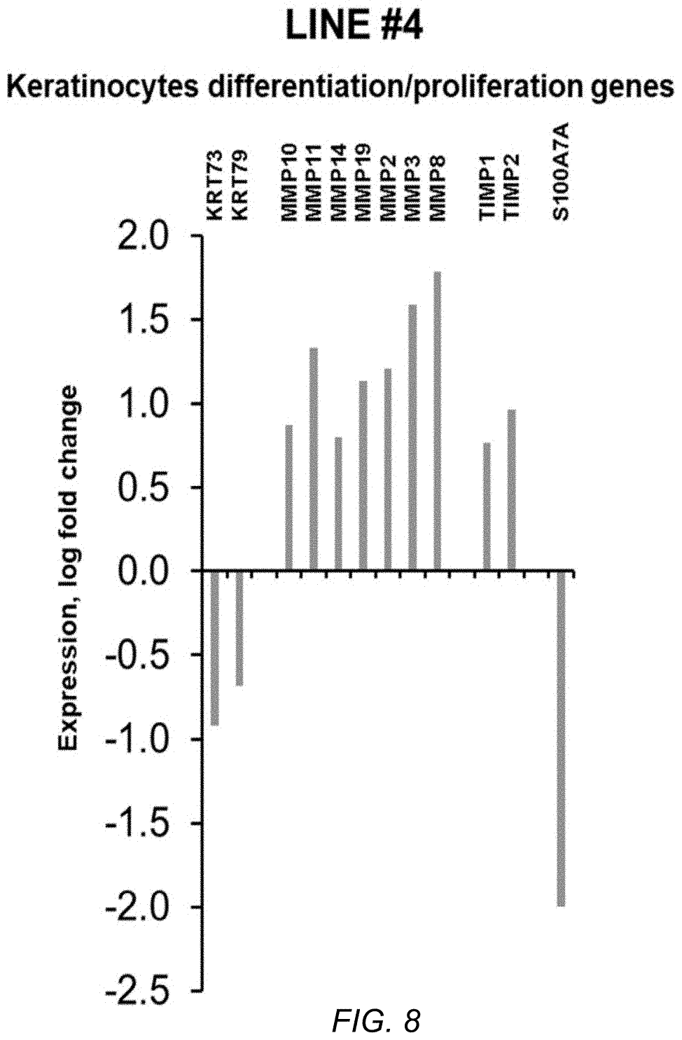

FIG. 8 shows the effect of extract #4: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

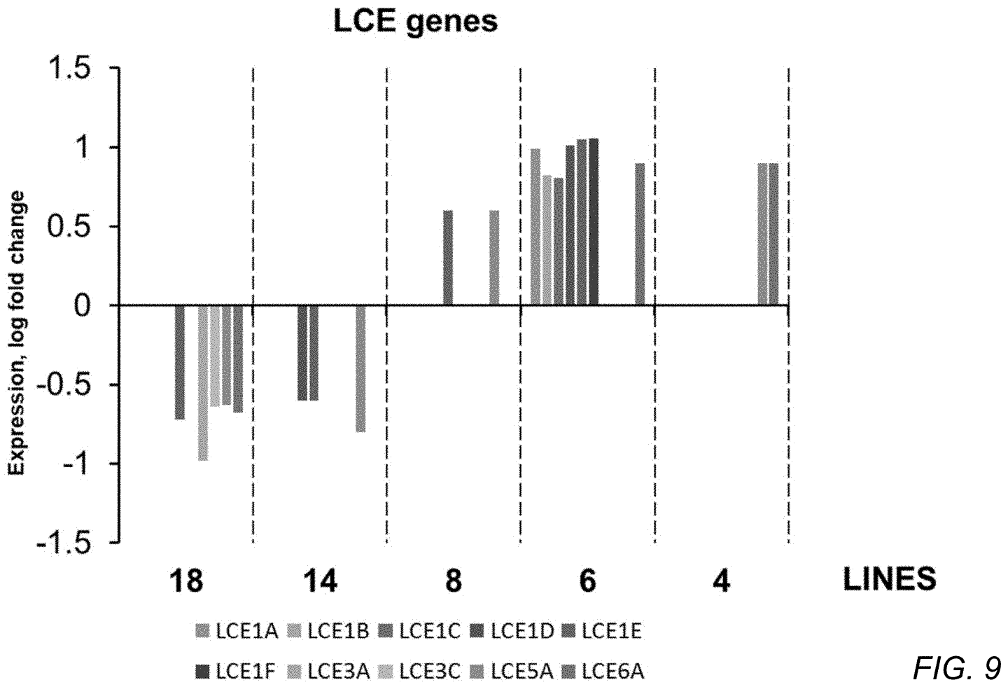

FIG. 9 shows the expression of LCE genes in PSOR skin upon treatment with extracts. Graphs show log₂ fold changes for differentially-expressed genes. No LCE genes were changed by extract #13.

FIG. 10A shows the EpiDermFt tissues and experimental set-up. EpiDermFT (Mattek) has normal skin tissue structure with differentiated dermis and epidermis and is constructed from human-derived epidermal keratinocytes and dermal fibroblasts. It exhibits in vivo-like growth and morphological characteristics whereby cells sustain differentiation and metabolic status similar to those of human epidermis. The model is widely used and accepted for platform for studying effects of various topical applications on skin [17]. FIG. 10B. Tissue insert in a well with medium. FIG. 10C. Scheme of experiments. To analyze the direct effects of extracts on skin, extracts were dissolved in coconut oil and applied directly to the skin model surface.

In tissue preparation step 1002-3D EpiDermFt tissues of normal skin epithelial tissues were used.

Further in an experimental test setup step 1004, 3D tissues were inserted in a well with medium. Tissues were equilibrated in EFT-400 for 24 h (overnight) then culture media EFT-400 was replaced and incubated for another 24 h.

In topical application step 1006, to analyze the effects of extracts on skin, extracts were dissolved in coconut oil and applied directly to the skin model surface for 24 h upon which the samples were taken for the analysis. Bioinformatics analysis of mRNA revealed changes in biological pathways associated with psoriasis.

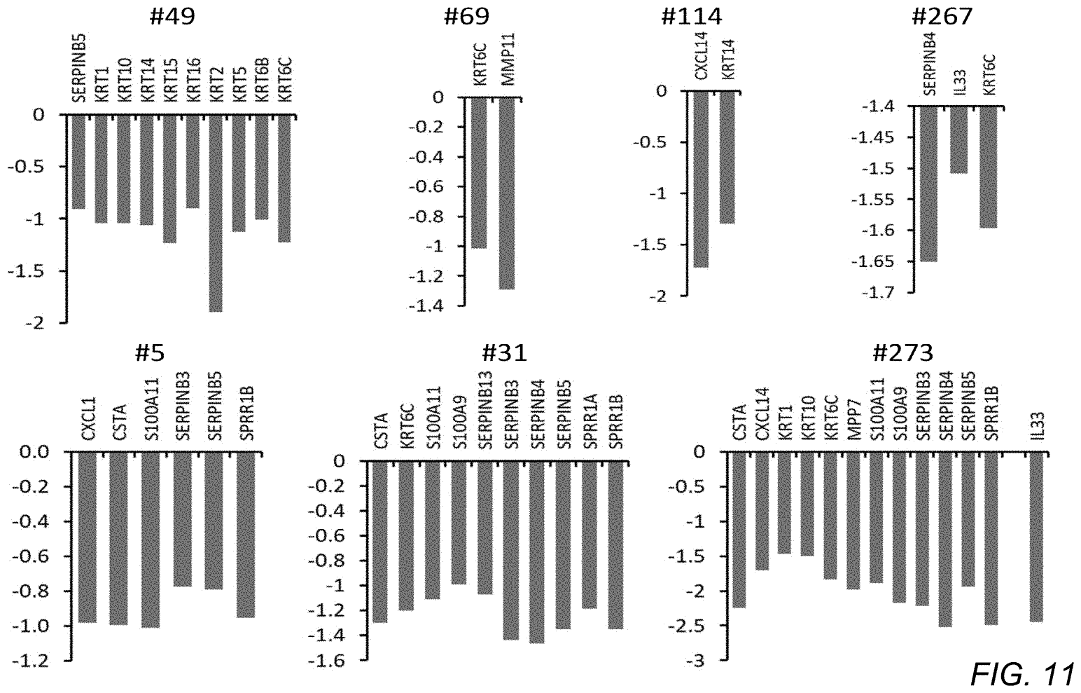

FIG. 11 shows the effect of extracts #49, #69, #114, #267, #5, #31, #273: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

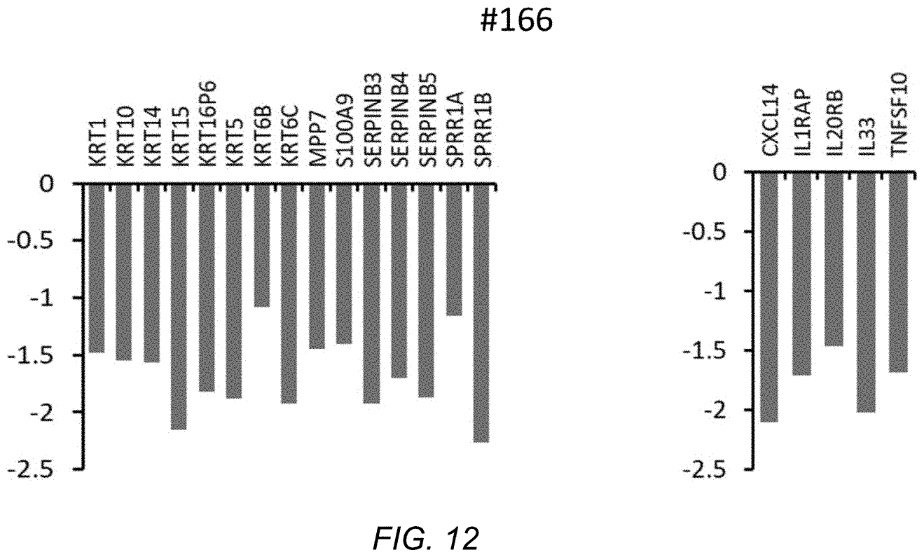

FIG. 12 shows the effect of extract #166: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

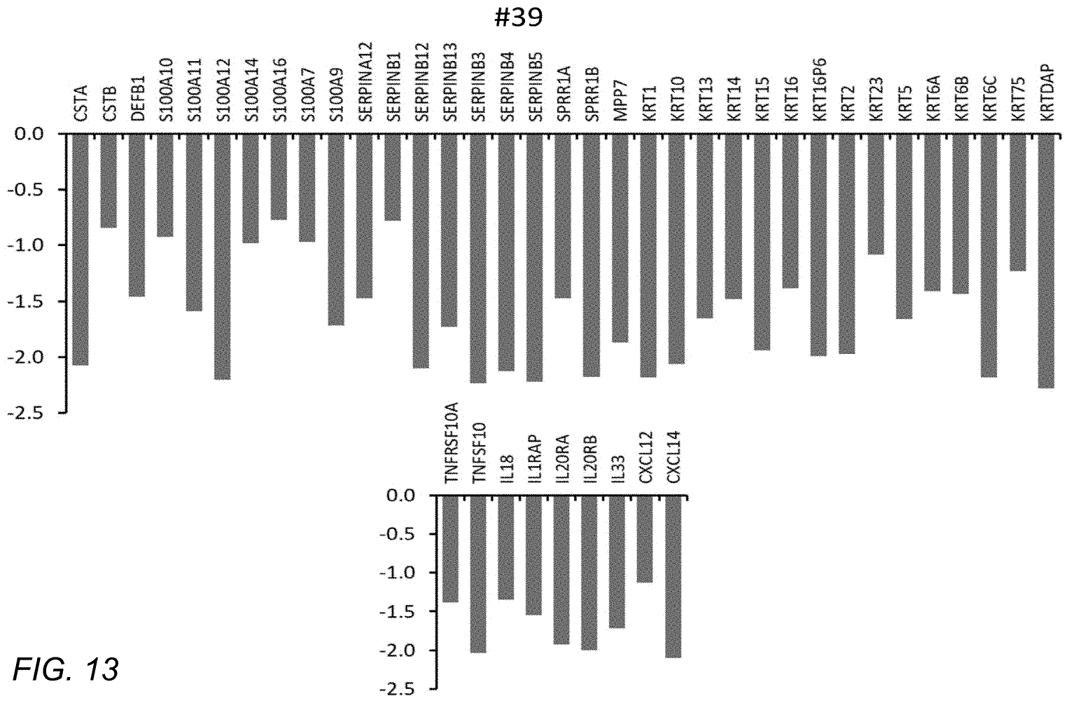

FIG. 13 shows the effect of extract #39: Differentially expressed Epidermal Differentiation Complex (EDC) genes involved in keratinization and proliferation and selected other genes associated with non-melanoma skin cancer or inflammatory skin diseases. Graphs show log₂ fold changes for differentially-expressed genes.

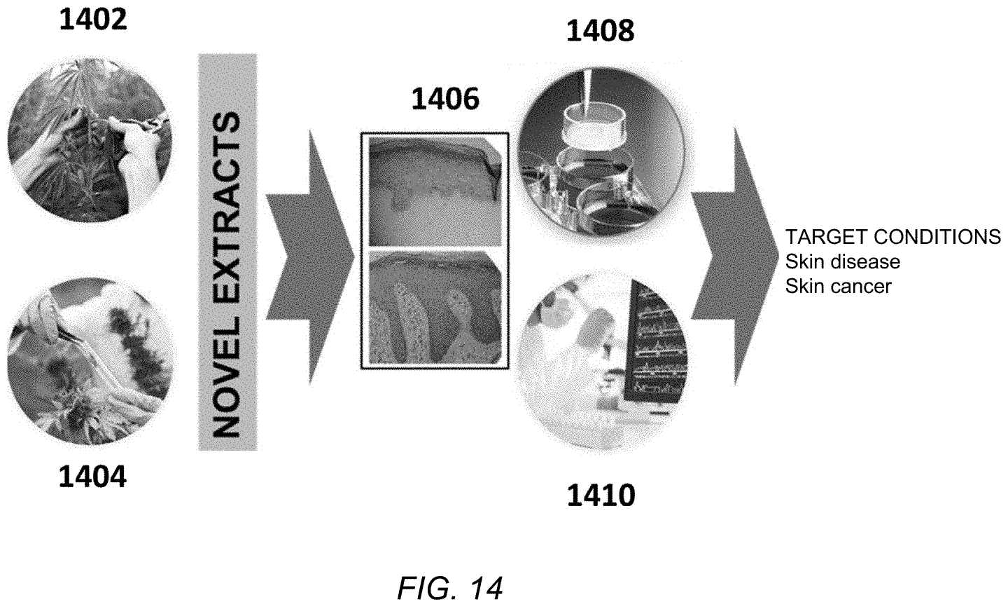

FIG. 14 shows a schematic summary of a methodology for finding active components for treating skin diseases,

Step 1402-a cultivar growing step. Around 250 unique marijuana and around 120 unique hemp cultivars were used to generate approximately 1,200 marijuana/marijuana, hemp/hemp and hemp/marijuana hybrids. Cultivars are typically grown in soil/vermiculite (2:1) mix. First, plants are grown under 16 h day, 8 h night for approximately 6 weeks when they were moved to another grow room and grown at 12 h day and 12 h night for another 6-8 weeks until they developed mature flowers. In both rooms, they were grown under the high pressure sodium (HPS) lights of ˜400 W/m2. Collected flowers were then tested for cannabinoids and terpenoids and those with most diversity in composition, or those that had highest amount of one or more cannabinoid or terpenoid or those that had the presence of unique terpenoids were used for breeding. The progeny of these crosses was then grown and further tested for cannabinoids/terpenoids as well as for growth parameters, such as height, response to nutrients, responses to pathogens, amongst others.

In some cases, these plants were then crossed again using siblings with similar traits (cannabinoids/terpenoids for example). The seeds of these cultivars (resulting from crosses) are stored at +4° C. in the fridge in the locked cage. Approximately 600 cultivars/cultivars with the best parameters, such as diversity of cannabinoids and terpenoids, plant growth vigor (germination rate, mutation time, yield of flowers, nutrients response, response to pathogens, size of flowers) and other features such as distinct smell for example were germinated and approximately 400 extracts were made.

In a preparing extracts step 1404, extracts from these cultivars were prepared.

Most organic solvents can be used for the extraction. In one experiment ethyl acetate was used. This should not be deemed as limiting. For extract preparation, 3 g of the powdered flower tissue were used in 100 ml of ethyl acetate in a 250 mL Erlenmeyer flask. The flasks were then wrapped with tin foil and shaken continuously (120 rpm) in an incubator at 21° C. overnight and in the dark. After overnight solvent extraction the extracts were filtered through cotton into a 100 ml round bottom flask. The extracts were concentrated to around 2-3 ml using a rotary vacuum evaporator. The extracts were then transferred to a tared 3 dram vial. The leftover solvent was evaporated to dryness in an oven overnight at 50° C. to eliminate the solvent completely. Mass of each extract was recorded, and the extracts were stored at −20° C. The stocks were prepared weighing a 3-6 mg of crude extract into a micro centrifuge tube. The crude extract was dissolved in DMSO (Dimethyl sulfoxide anhydrous) to reach 60 mg/mL final concentration and stored at −20° C. Around 400 solvent-based crude extracts of flowers were thus generated.

In a developing a skin test step 1406, psoriasis tissue model (SOR-300-FT) or normal 3D EpiDermFt skin epithelial tissues were used.

In an extract testing step 1408, tissues were exposed to extracts from various cultivars.

In bioinformatics analysis step 1410, tissues samples are used for mRNA sequencing and bioinformatics analysis of pathways related to psoriasis and novel treatments for skin conditions are identified.

Described herein is a method for treating skin disorders including, but is not limited to the steps of: 1) preparation of new cannabis extracts, 2) exposing normal and disease skin tissue models to novel extracts and 3) modulating the gene expression to cause a reduction of a disease state, or prevent an increase in the disease state in the skin tissues.

Treatments of psoriasis skin with extracts of new cannabis lines (#4, #6, #8, #13, #14, #18, #5, #31, #39, #49, #69, #114, #166, #267, #273) significantly affected gene expression in the 3D skin, leading to down-regulation of genes and pathways involved in keratinization and proliferation, inflammation, immunity and autoimmunity, ECM degradation (FIGS. 3-9, 11-13). Identified lines with the best anti-psoriasis properties were detected, based on the expression of genes that are associated with molecular etiology and pathogenesis of skin diseases. These lines included #18, #8, #6, and #13, and lines #39, # 166, #49, #31, and #273 (FIGS. 3-9, 11-13).

In a more specific embodiment, there is provided a method for treating psoriasis, eczema, inflammatory skin diseases and NMSC that includes the steps of exposing a patient to novel cannabis extracts, resulting in a reduction in the disease manifestation in the patient.

In some embodiments, extracts of new cannabis lines are applied to a human patient. In other embodiments, they may be applied to human skin tissue, artificial human skin tissue, or even animal skin tissue to modulate gene expression leading to a reduction, or at least prevent an increase in a disease state.

Among those genes down-regulated by new extracts are members of the Epidermal Differentiation Complex (EDC), including Small Proline Rich Region (SPRR), Late Cornified Envelope (LCE), and S100 genes. Other genes down-regulated by extracts are inflammatory cytokines and chemokines, members of interferon pathway, members of the human β-defensin family, and members of the serine protease inhibitor (serpin) genes. Many of these genes are up-regulated in skin disorders such psoriasis, atopic dermatitis and skin cancers and are transcriptomic biomarkes of psoriasis and skin diseases. Furthermore, the EDC gene S100P is known to be up-regulated in many tumor types, acting as a proliferative and anti-apoptotic agent.

In some situations, epidermal differentiation complex (EDC) genes (late cornified envelope (LCE) genes subfamily) may be down-regulated in certain disease states. Some of these genes known to be down-regulated in a certain disease state include, but are not limited to LCE1A, LCE1B, LCE1C, LCE1D, LCE1E, LCE1F and LCE5A. Exposure to extracts may result in up-regulation of these genes, leading to a decrease in the disease state (also see FIGS. 9). Other LCE genes are up-regulated in certain disease state, these include LCE3A, LCE3C, LCE6A, and exposure to extracts resulted in down-regulation of these genes leading to a decrease in the disease state (FIG. 9).

As used herein, “sequence identity” or “identity” in the context of two nucleic acid or protein sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

In addition, new extracts may modulate genes and proteins sharing a sequence identity or substantial sequence identity to those genes and proteins listed herein.

The presented Examples (FIGS. 2-14) are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

EDC complex genes are responsible for terminal differentiation of keratinocytes and regulation of epidermal barrier function, as well as for skin immune and inflammation responses. Up-regulation of EDC genes leads to increased proliferation and differentiation of keratinocytes commonly observed in inflammatory skin disorders such as psoriasis and atopic dermatitis, as well as in skin cancers. The 1q21 locus has been identified as one of the key psoriasis susceptibility loci (PSORS4) in clinical genome-wide linkage studies. Of the EDC genes that were down-regulated by extracts many, including S100A12, S100A7,S100A7A, S100A8, S100A9, SPRR2B,SPRR2D, SPRR2F, SPRR2G, SPRR3, SPRR4, LCE3 genes, are up-regulated in psoriasis (FIGS. 3-8, 11-13).

Enhanced expression of multiple EDC genes, including members of the SPRR family, S100 genes that encode for calcium-binding proteins, and multiple cornified envelope genes, has been identified as a hallmark of cutaneous squamous cell carcinomas (SCC).

Of these SCC-associated EDC genes, three, including S100A12, SPRR2B, SPRR3 are down-regulated by extracts (FIG. 3-8, 11-13). Down regulation of these genes by extracts may open new avenues for targeted treatments for psoriasis and SCC.

Besides EDC genes, extracts affected numerous other genes involved in psoriasis, atopic dermatitis and other inflammatory skin diseases, as well as in many cancer types, such as aggressive oral squamous cell carcinoma (OSCC) and non-melanoma skin cancer (SCC and basal cell carcinoma (BCC)). Down-regulation of matrix metalloproteinases (MMPs) was observed. These are involved in the structural changes of the epidermis via the modification of intracellular contacts and the composition of the extracellular matrix, promoting angiogenesis in the dermal blood vessels and the infiltration of immune cells. MMPs are also upregulated in skin cancers. Extracts potently down-regulated MMPs, resulting in decrease of the disease state (FIGS. 3, 5, 6, and 7).

Extracts also profoundly down-regulated pro-inflammatory genes—CCL8, CXCL5, CXCL1, CXCL6, CXCL14, CXCL8, IL32, IL23A, GJB2 and others which are usually over-expressed in psoriatic skin, and in cancers (FIGS. 3-8, 11-13).

Additionally, extracts significantly lowered mRNA expression of human (3-defensins, especially DEFB4—a known psoriasis biomarker. Enhanced expression of β-defensins is associated with non-melanoma skin cancer (basal cell carcinoma (BCC) and SCC), and OSCC, as well as psoriasis.

Exposure extracts also suppressed levels of serine proteinase inhibitor (serpin) genes that map to the serpin superfamily locus at chromosome 18q21.3, once again demonstrating special affinity to EDC locus genes. Extracts down-regulated the S100P gene that is related in function to the S100A family of genes of EDC and encodes for a member of the Ca²⁺-binding proteins family. Over-expression of S100P has been shown to act as a proliferative and anti-apoptotic factor in many tumor types.

In summary, the present invention demonstrates that extracts of new cannabis lines have profound impact on global gene expression in psoriasis. The observed extracts-induced changes in expression of EDC genes and other genes implicated in non-melanoma skin cancers and inflammatory skin disorders such as psoriasis, suggests the potential application of the extracts of new lines for treatment aimed at normalizing the expression of these disease-related genes. Extract efficacy can be further improved by adding additional CBD, CBG, CBN or terpenes.

### Dosage Forms

The compositions of the present invention may be provided in any suitable dosage form. According to some embodiments, the dosage form is an oral dosage form. Oral dosage forms comprise liquids (solutions, suspensions, and emulsions), semi-solids (pastes), and solids (tablets, capsules, powders, granules, premixes, and medicated blocks).

Some examples of oral dosage forms in the art include, WO90/04391, which discloses an oral dosage form of omega-3 polyunsaturated acids to overcome the problems of diseases. It is known to supply said acids in soft gelatine capsule shells.

EP 2 240 581 B1 discloses a gelatine capsule for pharmaceutical use with a controlled release of active ingredients and a process for the preparation of said gelatine capsules. During said process xylose is added to the liquid gelatine from which afterwards gelatine capsules are formed. Gelatine capsules manufactured according to the process provide retarded release of active ingredients.

U.S. Pat. No. 7,264,824 discloses and oral dosage form for food and food supplements, as well as dietetics comprising polyunsaturated acids in a xylose-hardened gelatine capsule with a retarded release time.

According to some embodiments of the present invention, the compositions described herein may be in a suspension or emulsion.

A suspension is a coarse dispersion of insoluble drug particles, generally with a diameter exceeding 1 μm, in a liquid (usually aqueous) medium. Suspensions are useful for administering insoluble or poorly soluble drugs/components or in situations when the presence of a finely divided form of the material in the GI tract is required. The taste of most drugs is less noticeable in suspension than in solution, due to the drug being less soluble in suspension. Particle size is an important determinant of the dissolution rate and bioavailability of drugs in suspension. In addition to the excipients described above for solutions, suspensions include surfactants and thickening agents. Surfactants wet the solid particles, thereby ensuring the particles disperse readily throughout the liquid. Thickening agents reduce the rate at which particles settle to the bottom of the container. Some settling is acceptable, provided the sediment can be readily dispersed when the container is shaken. Because hard masses of sediment do not satisfy this criterion, caking of suspensions is not acceptable.

An emulsion is a system consisting of 2 immiscible liquid phases, one of which is dispersed throughout the other in the form of fine droplets; droplet diameter generally ranges from 0.1-100 μm. The 2 phases of an emulsion are known as the dispersed phase and the continuous phase. Emulsions are inherently unstable and are stabilized through the use of an emulsifying agent, which prevents coalescence of the dispersed droplets. Creaming, as occurs with milk, also occurs with pharmaceutical emulsions. However, it is not a serious problem because a uniform dispersion returns upon shaking. Creaming is, nonetheless, undesirable because it is associated with an increased likelihood of the droplets coalescing and the emulsion breaking. Other additives include buffers, antioxidants, and preservatives. Emulsions for oral administration are usually oil (the active ingredient) in water, and facilitate the administration of oily substances such as castor oil or liquid paraffin in a more palatable form.

A paste is a 2-component semi-solid in which drug is dispersed as a powder in an aqueous or fatty base. The particle size of the active ingredient in pastes can be as large as 100 μm. The vehicle containing the drug may be water; a polyhydroxy liquid such as glycerin, propylene glycol, or polyethylene glycol; a vegetable oil; or a mineral oil. Other formulation excipients include thickening agents, cosolvents, adsorbents, humectants, and preservatives. The thickening agent may be a naturally occurring material such as acacia or tragacanth, or a synthetic or chemically modified derivative such as xanthum gum or hydroxypropylmethyl cellulose. The degree of cohesiveness, plasticity, and syringeability of pastes is attributed to the thickening agent. It may be necessary to include a cosolvent to increase the solubility of the drug. Syneresis of pastes is a form of instability in which the solid and liquid components of the formulation separate over time; it is prevented by including an adsorbent such as microcrystalline cellulose. A humectant (eg, glycerin or propylene glycol) is used to prevent the paste that collects at the nozzle of the dispenser from forming a hard crust. Microbial growth in the formulation is inhibited using a preservative. It is critical that pastes have a pleasant taste or are tasteless.

A tablet consists of one or more active ingredients and numerous excipients and may be a conventional tablet that is swallowed whole, a chewable tablet, or a modified-release tablet (more commonly referred to as a modified-release bolus due to its large unit size). Conventional and chewable tablets are used to administer drugs to dogs and cats, whereas modified-release boluses are administered to cattle, sheep, and goats. The physical and chemical stability of tablets is generally better than that of liquid dosage forms. The main disadvantages of tablets are the bioavailability of poorly water-soluble drugs or poorly absorbed drugs, and the local irritation of the GI mucosa that some drugs may cause.

A capsule is an oral dosage form usually made from gelatin and filled with an active ingredient and excipients. Two common capsule types are available: hard gelatin capsules for solid-fill formulations, and soft gelatin capsules for liquid-fill or semi-solid-fill formulations. Soft gelatin capsules are suitable for formulating poorly water-soluble drugs because they afford good drug release and absorption by the GI tract. Gelatin capsules are frequently more expensive than tablets but have some advantages. For example, particle size is rarely altered during capsule manufacture, and capsules mask the taste and odor of the active ingredient and protect photolabile ingredients.

A powder is a formulation in which a drug powder is mixed with other powdered excipients to produce a final product for oral administration. Powders have better chemical stability than liquids and dissolve faster than tablets or capsules because disintegration is not an issue. This translates into faster absorption for those drugs characterized by dissolution rate-limited absorption. Unpleasant tastes can be more pronounced with powders than with other dosage forms and can be a particular concern with in-feed powders, in which it contributes to variable ingestion of the dose. Moreover, sick animals often eat less and are therefore not amenable to treatment with in-feed powder formulations. Drug powders are principally used prophylactically in feed, or formulated as a soluble powder for addition to drinking water or milk replacer. Powders have also been formulated with emulsifying agents to facilitate their administration as liquid drenches.

A granule is a dosage form consisting of powder particles that have been aggregated to form a larger mass, usually 2-4 mm in diameter. Granulation overcomes segregation of the different particle sizes during storage and/or dose administration, the latter being a potential source of inaccurate dosing. Granules and powders generally behave similarly; however, granules must deaggregate prior to dissolution and absorption.

A premix is a solid dosage form in which an active ingredient, such as a coccidiostat, production enhancer, or nutritional supplement, is formulated with excipients. Premix products are mixed homogeneously with feed at rates (when expressed on an active ingredient basis) that range from a few milligrams to ˜200 g/ton of food/beverage The density, particle size, and geometry of the premix particles should match as closely as possible those of the feed in which the premix will be incorporated to facilitate uniform mixing. Issues such as instability, electrostatic charge, and hygroscopicity must also be addressed. The excipients present in premix formulations include carriers, liquid binders, diluents, anti-caking agents, and anti-dust agents. Carriers, such as wheat middlings, soybean mill run, and rice hulls, bind active ingredients to their surfaces and are important in attaining uniform mixing of the active ingredient. A liquid binding agent, such as a vegetable oil, should be included in the formulation whenever a carrier is used. Diluents increase the bulk of premix formulations, but unlike carriers, do not bind the active ingredients. Examples of diluents include ground limestone, dicalcium phosphate, dextrose, and kaolin. Caking in a premix formulation may be caused by hygroscopic ingredients and is addressed by adding small amounts of anti-caking agents such as calcium silicate, silicon dioxide, and hydrophobic starch. The dust associated with powdered premix formulations can have serious implications for both operator safety and economic losses, and is reduced by including a vegetable oil or light mineral oil in the formulation. An alternate approach to overcoming dust is to granulate the premix formulation.

A medicated block is a compressed feed material that contains an active ingredient, such as a drug, anthelmintic, surfactant (for bloat prevention), or a nutritional supplement, and is commonly packaged in a cardboard box. Ruminants typically have free access to the medicated block over several days, and variable consumption may be problematic. This concern is addressed by ensuring the active ingredient is nontoxic, stable, palatable, and preferably of low solubility. In addition, excipients in the formulation modulate consumption by altering the palatability and/or the hardness of the medicated block. For example, molasses increases palatability and sodium chloride decreases it. Additionally, the incorporation of a binder such as lignin sulfonate in blocks manufactured by compression or magnesium oxide in blocks manufactured by chemical reaction, increases hardness. The hygroscopic nature of molasses in a formulation may also impact the hardness of medicated blocks and is addressed by using appropriate packaging.

In another embodiment, the composition of the present invention is in a chewable oral dosage form. In another embodiment, the chewable oral dosage form is a chewable tablet. In another embodiment, the chewable tablet of the invention is taken slowly by chewing or sucking in the mouth. In another embodiment, the chewable tablet of the invention enables the dried cannabis extracts contained therein to be orally administered without drinking.

According to some embodiments of the present invention, the composition may comprise any suitable flavor or combination of flavors.

The composition may further comprise other additives, coloring, emulsifiers. The flavors and additives may be of a natural, semi-synthetic, synthetic source or combinations thereof.

In another embodiment of the present invention, the composition further comprises fructose, sorbitol, microcrystalline cellulose, magnesium stearate, or any combination thereof. In another embodiment, the composition further comprises chamomile. In another embodiment, the composition further comprises ginger. In another embodiment, the composition further comprises peppermint. In another embodiment, the composition further comprises anise. In another embodiment, the composition further comprises fennel. In another embodiment, the composition further comprises thyme. In another embodiment, the composition further comprises Arsenicum album. In another embodiment, the composition further comprises Carbo vegetabilis. In another embodiment, the composition further comprises Ignatia, homeopathic ipecac. In another embodiment, the composition further comprises Nux vomica. In another embodiment, the composition further comprises Zingiber officinale.

In another embodiment, the composition of the present invention is in the form of a chewing gum product. In another embodiment, chewing gum compositions contemplated by the present invention comprise all types of sugar and sugarless chewing gums and chewing gum formulations known to those skilled in the art, including regular and bubble gum types. In another embodiment, chewing gum compositions of the invention comprise a chewing gum base, a modifier, a bulking agent or sweetener, and one or more other additives such as, flavoring agents, colorants and antioxidants. In another embodiment, the modifying agents are used to soften, plasticize and/or compatibilize one or more of the components of the gum base and/or of the formulation as a whole.

In another embodiment, the present invention provides a soft, chewable dosage form which is pliable and chewy, yet dissolves quickly in the mouth, has a long shelf life, contains little moisture which improves stability and decreases the tendency for the dosage form to dry out, does not require cooking or heating as part of the manufacturing process. In another embodiment, the dosage form is used as a matrix for dried cannabis extracts.

In another embodiment, the chewable tablet of the invention comprises a metal salt such as calcium, magnesium, aluminum salt, or any mixture thereof. In another embodiment, the chewable tablet of the invention comprises hydroxyalkyl cellulose. In another embodiment, the chewable tablet of the invention comprises low viscosity hydroxyalkyl cellulose. In another embodiment, the chewable tablet of the invention comprises high viscosity hydroxyalkyl cellulose.

In another embodiment, the chewable tablet of the invention comprises various additives. In another embodiment, the chewable tablet of the invention comprises sweeteners. In another embodiment, the chewable tablet of the invention comprises acidic ingredients. In another embodiment, the chewable tablet of the invention comprises taste correctives. In another embodiment, the chewable tablet of the invention comprises polymeric compounds. In another embodiment, the chewable tablet of the invention comprises essential oils.

In another embodiment, the chewable tablet of the invention is a soft tablet. In another embodiment, the chewable tablet of the invention is made in a state of soft candy. In another embodiment, the chewable tablet of the invention is made in a state of jelly.

In another embodiment, the chewable tablet of the invention comprises a core comprising the vitamins of the invention. In another embodiment, the chewable tablet of the invention comprises an outer layer wrapping the core which is made up of chewable base such as a gum, a soft candy or a caramel.

In another embodiment, the compositions of the present invention may be provided in any suitable food of a solid, semi-solid or liquid form.

The preparation of pharmaceutical compositions that contain a dried cannabis extract, for example by mixing, granulating, or tablet-forming processes, is well understood in the art. The dried cannabis extracts are often mixed with excipients that are pharmaceutically acceptable and compatible with the active ingredient. For oral administration, the active ingredients of compositions of the present invention are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions.

In another embodiment, additional methods of administering the dried cannabis extracts, or compound(s) isolated therefrom, of the invention comprise injectable dosage forms. In another embodiment, the injectable is administered intraperitoneally. In another embodiment, the injectable is administered intramuscularly. In another embodiment, the injectable is administered intradermally. In another embodiment, the injectable is administered intravenously. Each possibility represents a separate embodiment of the present invention.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, additional methods of administering the dried cannabis extracts of the invention comprise dispersions, suspensions or emulsions. In another embodiment, the dispersion, suspension or emulsion is administered orally. In another embodiment, the solution is administered by infusion. In another embodiment, the solution is a solution for inhalation. Each possibility represents a separate embodiment of the present invention.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active compound agent over a period of time. Each possibility represents a separate embodiment of the present invention.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs. Each possibility represents a separate embodiment of the present invention.

In another embodiment, the pharmaceutical compositions provided herein are controlled-release compositions, i.e. compositions in which the active compounds are released over a period of time after administration. Controlled- or sustained-release compositions include formulation in lipophilic depots (e.g. fatty acids, waxes, oils). In another embodiment, the composition is an immediate-release composition, i.e. a composition in which all the active compound is released immediately after administration. Each possibility represents a separate embodiment of the present invention.

In another embodiment, the pharmaceutical composition is delivered in a controlled release system. In another embodiment, the agents are administered using intravenous infusion, an implantable osmotic pump, a transdermal patch, liposomes, or other modes of administration. In another embodiment, a pump is used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the therapeutic target, thus requiring only a fraction of the systemic dose (see, e.g., Goodson, in Medical Applications of Controlled Release, supra, vol. 2, pp. 115-138 (1984); and Langer R, Science 249: 1527-1533 (1990). Each possibility represents a separate embodiment of the present invention.

The compositions also include, in another embodiment, incorporation of the active materials into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance. Each possibility represents a separate embodiment of the present invention.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors. Each possibility represents a separate embodiment of the present invention.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications also increase, in another embodiment, the compound's solubility in aqueous solution, eliminate aggregation, enhance the physical and chemical stability of the compound, and greatly reduce the immunogenicity and reactivity of the compound. In another embodiment, the desired in vivo biological activity is achieved by the administration of such polymer-compound abducts less frequently or in lower doses than with the unmodified compound. Each possibility represents a separate embodiment of the present invention.

The compositions of the present invention may comprise one or more additional components may further include an additional component selected from the group consisting of an anti-static agent, a buffering agent, a bulking agent, a chelating agent, a colorant, a diluent, a dye, an emollient, a fragrance, an occlusive agent, a pH-adjusting agent, a preservative, and a vitamin

The compositions of the present invention may comprise one or more additional active agents, selected from the group consisting of active herbal extracts, analgesics, anti-allergic agents, anti-aging agents, anti-bacterials, antibiotic agents, anticancer agents, antidandruff agents, antidepressants, anti-dermatitis agents, anti-edemics, antihistamines, anti-helminths, anti-hyperkeratolyte agents, anti-inflammatory agents, anti-irritants, anti-microbials, anti-mycotics, anti-proliferative agents, antioxidants, anti-wrinkle agents, anti-pruritics, antiseptic agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, keratolytic agents, lactams, metals, metal oxides, mitocides, neuropeptides, non-steroidal anti-inflammatory agents, oxidizing agents, photodynamic therapy agents, retinoids, sanatives, scabicides, self-tanning agents, skin whitening agents, vasoconstrictors, vasodilators, vitamins, vitamin D derivatives and wound healing agents.

According to some embodiments, the composition may comprise one or more anti-oxidants/radical scavengers. The anti-oxidant/radical scavenger may be selected from butylated hydroxy benzoic acids and their salts, coenzyme Q10, coenzyme A, gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

In one embodiment, the term “treating” refers to curing a disease. In another embodiment, “treating” refers to preventing a disease. In another embodiment, “treating” refers to reducing the incidence of a disease. In another embodiment, “treating” refers to ameliorating symptoms of a disease. In another embodiment, “treating” refers to inducing remission. In another embodiment, “treating” refers to slowing the progression of a disease.

The references cited herein teach many principles that are applicable to the present invention. Therefore the full contents of these publications are incorporated by reference herein where appropriate for teachings of additional or alternative details, features and/or technical background.

It is to be understood that the invention is not limited in its application to the details set forth in the description contained herein or illustrated in the drawings. The invention is capable of other embodiments and of being practiced and carried out in various ways. Those skilled in the art will readily appreciate that various modifications and changes can be applied to the embodiments of the invention as hereinbefore described without departing from its scope, defined in and by the appended claims.

### REFERENCES

1. Karimkhani, C., et al., Global Skin Disease Morbidity and Mortality: An

Update From the Global Burden of Disease Study 2013. JAMA Dermatol, 2017. 153(5): p. 406-412.

2. Reed, B. and M. S. Blaiss, The burden of atopic dermatitis. Allergy Asthma Proc, 2018. 39(6): p. 406-410.

3. Igawa, K., Future trends in the treatment of atopic dermatitis. Immunol Med, 2019. 42(1): p. 10-15.

4. Rendon, A. and K. Schakel, Psoriasis Pathogenesis and Treatment. Int J Mol Sci, 2019. 20(6).

5. Oestreicher, J. L., et al., Molecular classification of psoriasis disease-associated genes through pharmacogenomic expression profiling. Pharmacogenomics J, 2001. 1(4): p. 272-87.

6. Ainali, C., et al., Transcriptome classification reveals molecular subtypes in psoriasis. BMC Genomics, 2012. 13: p. 472.

7. Quekenborn-Trinquet, V., et al., Gene expression profiles in psoriasis: analysis of impact of body site location and clinical severity. Br J Dermatol, 2005. 152(3): p. 489-504.

8. Hawkes, J. E., et al., Discovery of the IL-23/IL-17 Signaling Pathway and the Treatment of Psoriasis. J Immunol, 2018. 201(6): p. 1605-1613.

9. Yang, L., et al., Nrf2 Promotes Keratinocyte Proliferation in Psoriasis through Up-Regulation of Keratin 6, Keratin 16, and Keratin 17. J Invest Dermatol, 2017. 137(10): p. 2168-2176.

10. Izuhara, K., et al., Squamous Cell Carcinoma Antigen 2 (SCCA2, SERPINB4): An Emerging Biomarker for Skin Inflammatory Diseases. Int J Mol Sci, 2018. 19(4).

11. Broome, A. M., D. Ryan, and R. L. Eckert, S100 protein subcellular localization during epidermal differentiation and psoriasis. J Histochem Cytochem, 2003. 51(5): p. 675-85.

12. Granata, M., et al., S100A7 in Psoriasis: Immunodetection and Activation by CRISPR technology. Methods Mol Biol, 2019. 1929: p. 729-738.

13. Duvetorp, A., et al., Observational study on Swedish plaque psoriasis patients receiving narrowband-UVB treatment show decreased S100A8/A9 protein and gene expression levels in lesional psoriasis skin but no effect on S100A8/A9 protein levels in serum. PLoS One, 2019. 14(3): p. e0213344.

14. Kjaer, T. N., et al., Resveratrol ameliorates imiquimod-induced psoriasis-like skin inflammation in mice. PLoS One, 2015. 10(5): p. e0126599.

15. Derakhshan, N. and M. Kazemi, Cannabis for Refractory Psoriasis-High Hopes for a Novel Treatment and a Literature Review. Curr Clin Pharmacol, 2016. 11(2): p. 146-7.

16. Rogers, H. W., et al., Incidence Estimate of Nonmelanoma Skin Cancer (Keratinocyte Carcinomas) in the U.S. Population, 2012. JAMA Dermatol, 2015. 151(10): p. 1081-6.

17. Niehues, H. and E. H. van den Bogaard, Past, present and future of in vitro 3D reconstructed inflammatory skin models to study psoriasis. Exp Dermatol, 2018. 27(5): p. 512-519.

1. A method for treating mammalian skin, the method comprising:
a) combining at least one marijuana or hemp cultivar and at least one other marijuana or hemp cultivar to form at least one Cannabis line;
b) extracting at least one compound from said at least one Cannabis line to form an extract; and
c) treating skin with at least one of said extract and said at least one compound in an effective amount to treat said skin.

2. A method according to claim 1, wherein said treating step induces modulation of gene expression in at least one of skin cells and skin tissue; and wherein said modulation of gene said expression results in a reduction of at least one of a skin disease state, an inflammatory state, a skin disorder state, a poor skin appearance state, a skin aging state and combinations thereof.

3. A method according to claim 1, wherein said at least one Cannabis line is selected from the group consisting of a marijuana/marijuana hybrid line, hemp/hemp hybrid line and hemp/marijuana hybrid line.

4. A method according to claim 3, wherein said at least one line is selected from the group consisting of designated lines #4, #6, #8, #13, #14 and #18, #5, #31, #39, #49, #69, #114, #166, #267, #273.

5. A method according to claim 1, wherein said extracting step comprises extracting flowers of said at least one Cannabis line.

6. A method according to claim 5, wherein said extracting step comprises extracting said at least one compound in at least one organic solvent.

7. A method according to claim 6, wherein said extracting step is performed at a temperature in the range of 15-to 60° C. and at a pressure in a range of −0.5 to 1.5 bar and wherein said at least one organic solvent comprises ethyl acetate.

8. A method according to claim 2, wherein said modulation of gene expression results in a reduction of a 0.1-3 log₂ fold reduction in at least one of an epidermal differentiation complex (EDC) gene, a keratinocyte differentiation gene and a keratinocyte proliferation gene, an inflammatory marker gene, an inflammation gene and combinations thereof.

9. A method according to claim 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT13, KRT15, KRT16P3, KRT 19, KRT23, KRT34, KRT37, KRT7, SPRR2B, SPRR2D, SPRR2F, SPRR2G, SPPP3, SPRR4, CSTA, CNFN, TNC, CHN3, RHCG, FABP3, FABP6, MMP1, MMP10, MMP2, MMP9, DEFB4A, S100A12, S100A7A, S100A8 and S100A9, and combinations thereof.

10. A method according to claim 9, wherein said at least one Cannabis line comprises line #18.

11. A method according to claim 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT10, SERPINB12, BTC ANDS100A7A and combinations thereof.

12. A method according to claim 11, wherein said at least one Cannabis line comprises line #14.

13. A method according to claim 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT 19, KRT37, KRT7, SPRR2A, SPRR2B, SPRR4, CNFN, RHCG, MMP10, DEFB4A, S100A7A, CCL8, IL23A and IL32 and combinations thereof.

14. A method according to claim 13, wherein said at least one Cannabis line comprises line #13.

15. A method according to claim 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT15, KRT 19, KRT7, KRT8, SERPIN F1, MMP10, MMP14, MMP2 MMP9, DEFB4A, S100A7A, CXCL1, CXCL14, CXCL15, CXCL6, and IL32 and combinations thereof.

16. A method according to claim 15, wherein said at least one Cannabis line comprises line #8.

17. A method according to claim 8, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT19, KRT42P, KRT17, DCN, CNFN, FABP3, MMP10, MMP17, MMP2, MMP3, MMP9, DEFB4A, S100A12, S100A7, S100A7A, S100A8, S100A9, IL13RA1, IL13RA2, IL1R1, IL1R2 IL1RL1, IL24, IL2RG, IL32, GJB2, CXCL1, CXCL5, CXCL6, CXCL8 and combinations thereof.

18. A method according to claim 17, wherein said at least one Cannabis line comprises line #6.

19. A method according to claim 2, wherein said modulation of gene expression results in at least one of an increase or an reduction in a 0.1-3 log₂ fold in at least one of an epidermal differentiation complex (EDC) gene, a keratinocyte differentiation gene and a keratinocyte proliferation gene, an inflammatory marker gene, an inflammation gene and combinations thereof.

20. A method according to claim 19, wherein said of at least one of said epidermal differentiation complex (EDC) gene, said keratinocyte differentiation gene and said keratinocyte proliferation gene, said inflammatory marker gene, said inflammation gene and combinations thereof, is selected from the group consisting of: KRT73, KRT79, MMP10, MMP11, MMP14, MMP19, MMP2, MMP3, MMP8, TIMP1, TIMP2 and S100A7A and combinations thereof.
